(19) United States
(12) Patent Application Publication
(10) Pub. No.: US 2011/0130838 A1
(43) Pub. Date: Jun. 2, 2011

(54) INTERVERTEBRAL IMPLANT AND INSTALLATION TOOL

(75) Inventor: Rudolf Morgenstern Lopez, Barcelona (ES)

(73) Assignee: Interventional Spine, Inc., Irvine, CA (US)

(21) Appl. No.: 12/994,736

(22) PCT Filed: May 21, 2009
(86) PCT No.: PCT/IB2009/005972

§ 371 Date: Feb. 10, 2011

Related U.S. Application Data
(60) Provisional application No. 61/176,460, filed on May 7, 2009.

(30) Foreign Application Priority Data
May 26, 2008 (ES) 200801551

(51) Int. Cl. A61F 2/44; A61B 17/56

(52) U.S. Cl. 623/17.16; 606/86 A

(57) ABSTRACT

An intervertebral implant (25), an installation tool (500), and related methods are provided for ensuring a minimum distance between two vertebrae. The implant (25) can comprise a pair of opposing body portions (1, 2) and an expansion component. The expansion component can rotate relative to the body portions (1, 2) in order to urge a head portion (4) thereof against one or more inclined contact surfaces of at least one of the body portions (1, 2). In this manner, the body portions (1, 2) can be separated, thereby increasing a height of the implant (25). The installation tool (500) can comprise a plurality of components that can be moved relative to each other to facilitate expansion or contraction of the implant (25).

### CROSS-REFERENCE TO RELATED APPLICATIONS

This application claims priority benefit under 35 U.S.C. §119(a) from Spanish Patent Application No. ES 200801551, filed May 26, 2008, and under 35 U.S.C. §119(e) from U.S. Provisional Application Ser. No. 61/176,460, filed on May 7, 2009, the entireties of the disclosures of each of which are hereby expressly incorporated herein by reference.

### BACKGROUND

1. Field of the Inventions

The present inventions relate to medical devices and, more particularly, to an intervertebral implant and an installation tool.

2. Description of the Related Art

The human spine is a flexible weight bearing column formed from a plurality of bones called vertebrae. There are thirty-three vertebrae, which can be grouped into one of five regions (cervical, thoracic, lumbar, sacral, and coccygeal). Moving down the spine, there are generally seven cervical vertebrae, twelve thoracic vertebrae, five lumbar vertebrae, five sacral vertebrae, and four coccygeal vertebrae. The vertebrae of the cervical, thoracic, and lumbar regions of the spine are typically separate throughout the life of an individual. In contrast, the vertebra of the sacral and coccygeal regions in an adult are fused to form two bones, the five sacral vertebrae which form the sacrum and the four coccygeal vertebrae which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. Thus, spinal fusion is the process by which the damaged disc is replaced and the spacing between the vertebrae is restored, thereby eliminating the instability and removing the pressure on neurological elements that cause pain.

Spinal fusion can be accomplished by providing an intervertebral implant between adjacent vertebrae to recreate the natural intervertebral spacing between adjacent vertebrae. Once the implant is inserted into the intervertebral space, osteogenic substances, such as autogenous bone graft or bone allograft, can be strategically implanted adjacent the implant to prompt bone ingrowth in the intervertebral space. The bone ingrowth promotes long-term fixation of the adjacent vertebrae. Various posterior fixation devices (e.g., fixation rods, screws etc.) can also be utilize to provide additional stabilization during the fusion process.

Recently, intervertebral implants have been developed that allow the surgeon to adjust the height of the intervertebral implant. This provides an ability to intra-operatively tailor the intervertebral implant height to match the natural spacing between the vertebrae. This reduces the number of sizes that the hospital must keep on hand to match the variable anatomy of the patients.

In many of these adjustable intervertebral implants, the height of the intervertebral implant is adjusted by expanding an actuation mechanism through rotation of a member of the actuation mechanism. In some intervertebral implants, the actuation mechanism is a screw or threaded portion that is rotated in order to cause opposing plates of the implant to move apart. In other implants, the actuation mechanism is a helical body that is counter-rotated to cause the body to increase in diameter and expand thereby.

Furthermore, notwithstanding the variety of efforts in the prior art described above, these intervertebral implants and techniques are associated with another disadvantage. In particular, these techniques typically involve an open surgical procedure, which results higher cost, lengthy in-patient hospital stays and the pain associated with open procedures.

Therefore, there remains a need in the art for an improved intervertebral implant. Preferably, the implant is implantable through a minimally invasive procedure. Further, such devices are preferably easy to implant and deploy in such a narrow space and opening while providing adjustability and responsiveness to the clinician.

### SUMMARY

While using minimally invasive procedures to deploy an intervertebral prostheses is generally advantageous, such procedures do have the disadvantages of generally requiring the device to be passed through a relatively small diameter passage or tube. In addition, deployment tools typically must also be deployed through the small diameter passage or tube.

As described, a typical intervertebral implant includes expansion members that are deployed to a fixed position and dimension. In this regard, according to at least one of the embodiments disclosed herein is the realization that the deployed implant is completely rigid, which is unnatural and affects the comfort of the patient's movements. In addition, many prior art intervertebral prostheses are not adjustable in height. In other words, a surgeon cannot precisely set the spacing between vertebrae secured by the implant.

Furthermore, after deploying the implant, extraction or positional adjustments using an minimally invasive procedures are potentially dangerous and can damage the tissue of the patient. These disadvantages can cause neuritis, among other complications. Nevertheless, it is generally common for a surgeon to have to relocate or remove the implant because the surgeon often has no means of knowing exactly where the implant is located.

Therefore, in accordance with at least one of the embodiments disclosed herein, there is provided an implant for use of intervertebral endoscope that overcomes the aforementioned drawbacks. For example, the implant can even be adjustable in height once installed, which allows the implant to be extracted or adjusted in the event of incorrect placement. Further, in some embodiments, the implant can allow for a degree of elasticity in the minimum separation of vertebrae.

More specifically, some embodiments disclosed herein comprise an intervertebral implant that can maintain a minimum distance between two joint vertebrae. The implant can comprise two expandable body portions and an expansion component. The two body portions can each have a general shape of a wedge. In some embodiments, each body portion can comprise a first surface configured to contact a vertebra and a second surface. In some embodiments, the second surface can be oriented obliquely relative to the first surface. Further, the second surface can be an inner surface that is inclined or slanted relative to the first surface.

In addition, the second surface of each body portion can be configured to allow the two body portions to be introduced against each other. In this regard, the body portions can comprise one or more structural components that allow the body portions to be interconnected or releasably mated. For example, each of the body portions can comprise one or more offset structures that allow the second surfaces of the body portions to traverse each other, such as by an interlinked or interweaving configuration. In such an embodiment, the second surfaces can be defined by top surfaces or planes defined by one or more raised structures. Further, the body portions can define one or more gaps or spaces adjacent to the one or more raised structures. In this regard, when the body portions are interlinked, one or more raised structures of one of the body portions can be received into one or more gaps or spaces of the other body portion such that the body portions can be at least partially interlinked with the second surfaces traversing each other.

Furthermore, in accordance with some embodiments, the expansion component of the implant can engage the body portions to facilitate separation of the body portions. In this regard, the expansion component of the implant can move along a longitudinal axis of the implant and cause one or both of the body portions to move in a direction transverse to the longitudinal axis of the implant so as to cause the body portions to move apart from each other. For example, in some embodiments, the expansion component can comprise a rounded or spheroid-shaped area that can engage or contact the second surface of the body portions. In certain embodiments, the expansion component can contact inclined second surfaces of the body portions to spread or urge the body portions apart.

The expansion component can comprise a head portion and a ram member. The head portion can be shaped as a spheroid, an ellipsoid, a cone, or as a pyramid having three or more sides, such as a triangular or square pyramid. Further, the head portion and the ram member can be formed separately from each other as individual components or can be formed as a unitary or monolithic piece. Accordingly, in an embodiment, the ram member can contact the head portion, and advancement of the ram member along the longitudinal axis can cause the head portion to move with the rain member relative to the body portions of the implant on thereby forcing the body portions apart.

In embodiments wherein the head portion of the expansion component is formed separately from the ram member of the expansion component, the implant can also comprise a confinement casing. The confinement casing can be configured to limit and/or prevent the movement of the head portion in a direction other than along the longitudinal axis of the implant. Accordingly, movement of the head portion caused by contact from the ram member can be confined to movement along the longitudinal axis. In this regard, motion of the ram member can be transferred efficiently and effectively to the head portion to cause the body portions to separate and cause a change in the height of the implant. Thus, in embodiments utilizing the confinement casing, the casing can prevent the head portion of the expansion component from exiting the activity area or space defined between the body portions.

In some embodiments, the confinement casing can comprise a channel. The channel can be configured to at least partially receive the ram member of the expansion component. For example, the channel can include one or more retention structures that can engage corresponding retention structures of the ram member. In such an embodiment, the retention structures of the channel can comprise one or more threads that threadably connect with threads of the ram member. In this regard, the engagement of retention structures of the channel with the retention structures of the rain member can not only provide an unlimited possibility of implant heights, but can also maintain the implant height against forces seeking to collapse the body portions into each other.

Additionally, the confinement casing can comprise a cap or lid element located on an end that is opposite the channel. For example, the confinement casing can be an elongate member with a first end and a second end. The channel can be formed in the first end of the confinement casing and the lid component can be disposed at the second end of the confinement casing. Moreover, the confinement casing can comprise a pair of sidewalls extending intermediate the lid component and the first end of the confinement casing. The pair of sidewalls can define a compartment therebetween into which the body portions can be at least partially received. In this regard, the compartment can be defined by the sidewalls, the lid component, and an end face of the channel. Accordingly, in such an embodiment, the expansion component can be disposed through the channel and extend into the compartment such that at least the head portion of the expansion component is disposed between body portions seated within the compartment.

One of the unique advantages of some embodiments is that the compartment of the confinement casing can be configured to guide or limit relative movement between the body portions. For example, the pair of sidewalls positioned along the sides of the compartment can prevent side-to-side relative motion between the body portions and guide vertical expansive or contractive relative movement between the body portions. Further, the lid component can prevent end-to-end relative motion between the body portions while also guiding the vertical expansive or contractive relative movement between the body portions. This advantageous configuration can thereby facilitate proper relative movement of the body portions and minimize the possibility of misalignment or dislocation of the body portions from their vertical relative movement.

The separation or height of the implant can be defined by external surfaces of the body portions. In turn, the separation between the external surfaces depends on the degree of penetration or axial displacement of the head portion which can be in contact with the second surfaces of the body portions. Further, the degree of penetration or axial displacement of the head portion between the body portions depends on the movement or progress of the ram member.

In accordance with some embodiments, the implant can comprise a height-limiting component. The height-limiting component can limit the relative motion between the body portions. For example, the height-limiting component can comprise one or more recesses or projections on at least one side of one or both of the body portions. The recesses or projections of the body portion(s) can engage corresponding projections or recesses formed on the confinement casing. For example, in an embodiment, the body portions can each comprise one or more recesses that engage corresponding protrusions formed along the sidewalls of the confinement casing. In use, such an embodiment can have a predetermined maximum implant height that is reached when the protrusions of the confinement casings engage an end of the recesses of the body portions, thus preventing further relative movement between the body portions.

As noted above, in some embodiments, the body portions can comprise structural components that allow the body portions to interlink. In accordance with such an embodiment, the structural components can limit one or more degrees of movement between the body portions. As such, when the body portions are interlinked, vertical movement can be permitted while horizontal movement is restricted. The structural components of the body portions can have a dual function. First, they can guide relative motion between the body portions. Second, they can ensure a minimum implant height or distance between body portions. Further, the structural components can form an internal wedge structure against which the head portion can act, allowing the implant height to be varied along a continuum of positions. Additionally, it is contemplated that the implant height can be varied along a plurality of discrete positions.

One of the unique advantages of embodiments of the implant is that the implant can avoid locking and can be easily adjustable and reversible. Reversibility greatly facilitates the placement of the implant. Moreover, the head portion can be used as a shock absorber. For example, the head portion can be made of a material that presents certain elastic properties, such as Teflon or nylon, which allows a degree of impact absorption, without compromising too much in maintaining the minimum separation between vertebrae.

In some embodiments, the implant can provide an elastic recovery element that interconnects the body portions. The elastic recovery element can be configured as an elastic mesh material or a rubber or elastic toroid, for example. In the event of a dislocation of the body portions relative to each other, the elastic recovery element can facilitate the relocation or return of the body portions to the pre-dislocation position.

For example, in an embodiment, the elastic recovery element can interconnect the body portions in a vertical direction and interact with the expansion component. In this regard, such an elastic recovery element can provide a vertical contracting force against the vertical expansion or separation of the body portions. In such an embodiment, the elastic recovery element can be placed in the space between body portions. In other embodiments, the expansion component can be configured to fit within the channel or compartment of the confinement casing.

In yet another embodiment, an intervertebral implant is provided for ensuring a minimum distance between two vertebrae. The implant can comprise a pair of body portions and an expansion component. The pair of body portions can each comprise an external surface and a contact surface that is oriented obliquely relative to the external surface. The body portions can each comprise at least one raised structure and at least one gap positioned adjacent to the raised structure. The raised structure can define a top surface that forms at least a portion of the contact surface of the body portion. The raised structures of each body portion and be insertable into the respective gaps of the other body portion such that the contact surfaces thereof define an internal wedge structure between the body portions.

Further, the expansion component can comprise a head portion and a ram member. The expansion component can be at least partially insertable between the body portions with the head portion positioned against the contact surfaces of the body portions. The ram member can be operative to urge the head portion against the contact surfaces such that movement of the head portion against the internal wedge structure causes the body portions to separate thereby increasing a height of the implant. In some embodiments, the expansion component can comprise one or more engagement structures for engaging with an expansion tool for rotating the expansion component. Further, the expansion component can comprise a threaded recess for engaging with the expansion tool for maintaining the expansion component in a given axial position relative to the tool during rotation of the expansion component.

In such an embodiment, the implant can further comprise a confinement casing to prevent the movement of the head portion of the expansion component in a direction transverse to a longitudinal axis of the implant. The confinement casing can comprise a channel configured to receive at least a portion of the ram member therein. Further, the confinement casing can comprise an elongate body having a lid at an end located distal to the channel and a compartment interposed between the lid and the channel. The compartment can be at least partially defined by a pair of sidewalls extending intermediate the lid and an end of the channel. The compartment can be configured to at least partially receive the body portions therein. Furthermore, the channel can be threaded, and the ram member can comprise at least one thread extending along an exterior surface thereof. In this regard, the ram member can be configured to threadingly engage the channel of the confinement casing. The casing can also comprise one or more engagement surfaces disposed at a proximal end of the casing, and the engagement surfaces can be configured to engage with an expansion tool for maintaining a rotational orientation of the implant with respect to at least a portion of the expansion tool.

Moreover, the ram member can move along a direction parallel to a longitudinal axis of the implant to urge the head portion against the contact surfaces of the body portions.

Additionally, some embodiments can comprise a recovery element extending between the body portions. The recovery element can be a mesh with elastic properties. For example, the recovery element can at least partially surround the body portions. Further, the recovery element can comprise an elastic rubber band.

In some embodiments, the implant can also comprise an expansion limiting system for limiting the expansion of the implant. The expansion limiting system can comprise a projection formed on one body portion that interferes with an end cap formed on the other body portion for limiting relative vertical motion between the body portions.

Further, the external surfaces of the body portions comprise one or more projections for promoting osseointegration of the surfaces with adjacent vertebrae.

In yet another embodiment, an intervertebral implant is provided for ensuring a minimum distance between two vertebrae. The implant can comprise a first body portion, a second body portion, and an expansion component. The first body portion can comprise a first external surface and a first contact surface. The first body portion can comprise at least one raised structure and at least one gap positioned adjacent to the raised structure. The second body portion can comprise a second external surface and a second contact surface that is oriented obliquely relative to the first external surface. The second body portion can comprise at least one raised structure and at least one gap positioned adjacent to the raised structure. The raised structure can define a top surface that forms at least a portion of the second contact surface of the body portion. In this regard, each raised structure of the first body portion can be insertable into the respective gap of the second body portion and each raised structure of the second body portion can be insertable into the respective gap of the first body portion such that the contact surfaces thereof define an internal wedge structure between the first body portion and the second body portion.

Further, the expansion component can comprise a head portion and a ram member. The expansion component can be at least partially insertable between the first body portion and the second body portion with the head portion positioned against the first and second contact surfaces. The ram member can be operative to urge the head portion against the first and second contact surfaces such that movement of the head portion against the internal wedge structure causes the first body portion to separate from the second body portion thereby increasing a height of the implant. In other embodiments, the expansion component can comprise a threaded recess for engaging with an expansion tool for maintaining the expansion component in a given axial position relative to the tool during rotation of the expansion component.

In some embodiments, the first contact surface of the first body portion can be oriented obliquely relative to the first external surface. Further, the head portion of the expansion component can be formed separately from the ram member. Furthermore, the head portion of the expansion component can comprise a generally spherical member. The head portion of the expansion component can be elastically deformable for providing a shock absorption capability to the implant. For example, the head portion is fabricated from one of nylon and Teflon. In addition, some embodiments can be implemented in which the head portion comprises at least one cavity for enhancing the shock absorption capability of the implant.

In other embodiments, the implant can comprise a confinement casing. The confinement casing can comprise a channel, a lid, and a compartment extending intermediate the channel and the lid. The channel can be configured to receive at least a portion of the ram member therein. The compartment can be at least partially defined by a pair of sidewalls extending intermediate the lid and an end of the channel. The compartment can be configured to at least partially receive the body portions therein. The confinement casing can be configured to align the body portions in a vertical direction and prevent movement of the expansion component in a direction transverse to a longitudinal axis of the implant.

In some embodiments, the channel can be threaded and the ram member can comprise at least one thread extending along an exterior surface thereof. In this regard, the ram member can be configured to threadingly engage the channel of the confinement casing. Further, the casing can comprise one or more engagement surfaces disposed at a proximal end of the casing. The engagement surfaces can be configured to engage with an expansion tool for maintaining a rotational orientation of the implant with respect to at least a portion of the expansion tool.

In accordance with yet another embodiment, an installation tool is provided for installing an implant. The tool can comprise a handle member, a first rotating member, and a second rotating member. The handle member can have a gripping component and an elongate tubular component extending from the gripping component. The tubular component can have a hollow bore and an engagement portion disposed at a distal end thereof. The engagement portion can have one or more protrusions for engaging at least a portion of a proximal end of an intervertebral implant to maintain a rotational orientation of the implant relative to the tubular component.

The first rotating member can have a first knob and an actuation component extending from the first knob. The actuation component can have a hollow bore and a rotational connector disposed at a distal end thereof. The actuation component can be configured to fit within the hollow bore of the tubular component of the handle member with the rotational connector being positioned adjacent to the engagement portion of the tubular component for engaging an expansion component of the implant for rotating the expansion component to expand or contract the implant.

Further, the second rotating member can have a second knob and a retention component extending from the second knob. The retention component can have a fastening portion disposed at a distal end thereof. The retention component can be configured to fit within the hollow bore of the actuation component of the first rotating member with the retention component being positioned adjacent to the rotational connector of the actuation component of the first rotational member for engaging the expansion component of the implant for maintaining an axial position of the implant relative to the handle member during rotation of the expansion component.

In some embodiments, the engagement portion of the tubular component of the handle member can comprise a pair of protrusions. Further, the pair of protrusions can be disposed on opposing sides of the tubular component with the implant being insertable therebetween. The rotational connector of the actuation component of the first rotating member can also comprise a pair of linear protrusions configured to be received in a slot of the expansion component of the implant. The tubular component of the actuation component and the retention component can also comprise generally cylindrical outer profiles. The retention component of the second rotating member can also be configured to draw the expansion component of the implant toward the actuation component of the first rotational member as the retention component engages the ram member. Furthermore, the fastening portion of the retention component can be threaded for threadably engaging the ram member of the implant.

In accordance with yet another embodiment, a method of implanting an expandable intervertebral implant is provided that can comprise: dilating a pathway to an intervertebral disc; removing the nucleus of an intervertebral disc to define a disc cavity; scraping vertebral end plates from within the disc cavity; and deploying an intervertebral implant in the disc cavity.

In some implementations of the method, the step of dilating can comprise: inserting a needle into the intervertebral disc; inserting a first dilator over the needle into the intervertebral disc; removing the needle; inserting a second dilator over the first dilator into the intervertebral disc; and removing the first dilator. Further, the method can comprise: inserting a first working sleeve over the second dilator to adjacent the intervertebral space; and removing the second dilator. Furthermore, the method can comprise: inserting a second working sleeve over the first working sleeve to adjacent the intervertebral space; and removing the first working sleeve.

Additionally, the step of removing the nucleus can comprise using a trephine tool. The step of removing the nucleus can also comprise using a punch tool. In some embodiments, the method can comprise drilling a hole into the intervertebral disc after dilation. In this regard, the step of drilling can comprise forming a hole in the intervertebral disc. The step of drilling can also comprise forming a hole in the vertebral end plates. Further, in some embodiments, the scraping step can comprise inserting a rasp into the intervertebral disc to scrape the vertebral end plates from within the disc cavity. Furthermore, the step of deploying the implant can comprise expanding the implant from approximately 9 mm to approximately 12.5 mm in height.

### BRIEF DESCRIPTION OF THE DRAWINGS

The abovementioned and other features of the inventions disclosed herein are described below with reference to the drawings of the preferred embodiments. The illustrated embodiments are intended to illustrate, but not to limit the inventions. The drawings contain the following figures:

FIG. 1 is a perspective view of an intervertebral implant, according to an embodiment of the present inventions.

FIG. 2 is an exploded perspective view of the implant of FIG. 1 and an expansion tool for adjusting a height of the implant, according to an embodiment.

FIG. 3 is a perspective view of the implant and the tool of FIG. 2 wherein the implant is in assembled state.

FIG. 4 is a perspective view of body portions of the implant of FIG. 1, according to an embodiment.

FIG. 5 is a side cross-sectional view of the implant and the tool of FIG. 3 wherein the tool is actuating an expansion component of the implant to increase the implant height, according to an embodiment.

FIG. 6 is a side cross-sectional view of the implant and the tool of FIG. 3 wherein the implant is in a collapsed state, according to an embodiment.

FIG. 7 is a perspective view of an intervertebral implant and an expansion tool, according to another embodiment.

FIG. 8 is an exploded perspective view of the implant and tool of FIG. 7.

FIG. 9 is a rear perspective view of the implant and the tool of FIG. 7, wherein the implant is in an expanded state.

FIG. 10 is a front perspective view of the implant and tool of FIG. 7, wherein the implant is in the expanded state.

FIG. 11 is a perspective cross-sectional view of the implant and tool of FIG. 7, wherein the implant is in the expanded state.

FIG. 12 is a perspective view of body portions of an intervertebral implant, wherein the body portions are in a collapsed state, in accordance with an embodiment.

FIG. 13 is a perspective view of the body portions of FIG. 12 having been expanded to an expanded state by actuation of a head portion of an expansion component of the implant, according to an embodiment.

FIG. 14 is a perspective view of a confinement casing of an intervertebral implant, according to an embodiment.

FIG. 15 is a front perspective view of a first body portion of an intervertebral implant, according to an embodiment.

FIG. 16 is a rear perspective view of the first body portion of FIG. 15.

FIG. 17 is a top view of the first body portion of FIG. 15.

FIG. 18 is a rear perspective view of a second body portion of an intervertebral implant, according to an embodiment.

FIG. 19 is a front perspective view of the second body portion of FIG. 18.

FIG. 20 is top view of the second body portion of FIG. 18.

FIG. 21 is a front perspective view of yet another embodiment of an intervertebral implant, wherein the implant is in a collapsed state.

FIG. 22 is a front perspective view of a first body portion of the intervertebral implant of FIG. 21.

FIG. 23 is a partial cross-sectional perspective view of the intervertebral implant of FIG. 21.

FIG. 24 is a perspective view of a confinement casing of the intervertebral implant of FIG. 21, according to an embodiment.

FIG. 25 is a perspective view of the intervertebral implant of FIG. 21, wherein the implant is in an expanded state.

FIG. 26 is a perspective view of first and second body portions and an expansion component of the intervertebral implant of FIG. 25, according to an embodiment.

FIG. 27 is a rear perspective view of first and second body portions being hingedly interconnected, according to an embodiment.

FIG. 28 is a side view of the first and second body portions of FIG. 27.

FIG. 29 is a perspective view of an expansion component, according to another embodiment.

FIG. 30 is a front perspective view of a first body portion, according to an embodiment.

FIG. 31 is a front perspective view of a second body portion, according to an embodiment.

FIG. 32 is a bottom perspective view of the second body portion of FIG. 31.

FIG. 33 is a perspective view of an installation tool and an intervertebral implant seated in a deployment portion of the tool, according to embodiments thereof.

FIG. 34 is a perspective view of the installation tool shown in FIG. 33.

FIG. 35 is a perspective view of first and second adjustment portions of the tool shown in FIG. 33, according to an embodiment.

FIG. 36 is a perspective view of the second adjustment portion of the tool shown in FIG. 33, according to an embodiment.

FIG. 37 is a cross-sectional top view of the installation tool and the intervertebral implant shown in FIG. 33 illustrating engagement between the tool and the implant, according to an embodiment.

FIG. 38 is a perspective view of the implant shown in FIG. 33.

FIG. 39 is an exploded view of the implant shown in FIG. 38, according to an embodiment.

FIG. 40 is a cross-sectional side view of the installation tool and the intervertebral implant shown in FIG. 33 illustrating engagement between the tool and the implant, wherein the implant is a collapsed state and portions of the implant and the tool are rotated 90° relative to that shown in FIG. 37.

FIG. 41 is a cross-sectional side view of the installation tool and the intervertebral implant shown in FIG. 33, wherein the implant is in an expanded state.

FIG. 42 is a top view of the installation tool and the intervertebral implant shown in FIG. 33.

FIG. 43 is a rear perspective view of the intervertebral implant and a distal engagement portion of the second adjustment portion of the installation tool, according to an embodiment.

FIG. 44 is a rear perspective view of an expansion component of the intervertebral implant, according to an embodiment.

FIG. 45 is a front perspective view of the expansion component shown in FIG. 44.

FIG. 46 is a bottom perspective view of a first body portion of the intervertebral implant, according to an embodiment.

FIG. 47 is a top perspective view of a second body portion of the intervertebral implant, according to an embodiment.

FIG. 48 is a cross sectional top view of the intervertebral implant shown in FIG. 38.

FIG. 49 illustrates a longitudinal cross-sectional view and an end view of a rasp tool in an unexpanded configuration, according to an embodiment.

FIG. 50 illustrates a longitudinal cross-sectional view and an end view of the rasp tool shown in FIG. 49, wherein the rasp tool is in an expanded configuration, according to an embodiment.

### DETAILED DESCRIPTION OF THE PREFERRED EMBODIMENTS

In accordance with certain embodiments disclosed herein, an improved intervertebral implant is provided that allows the clinician to insert the intervertebral implant through a minimally invasive procedure. For example, in one embodiment, one or more intervertebral implants can be inserted percutaneously to reduce trauma to the patient and thereby enhance recovery and improve overall results of the surgery. By minimally invasive, Applicant means a procedure performed percutaneously through an access device in contrast to a typically more invasive open surgical procedure. Such access devices typically provide an elongated passage that extends percutaneously through the patient to the target site. Examples of such access devices include, but are not limited to, endoscopes and the devices described in U.S. Patent Application Nos. 2006-0030872 and 2005-0256525 and U.S. Pat. Nos. 6,793,656 and 7,223,278, the entirety of these patent applications and patents are hereby incorporated by reference herein.

In some embodiments, the intervertebral implant can ensure a minimum distance between adjacent vertebrae (a function that a healthy individual's intervertebral disc can performs naturally). Because embodiments of the intervertebral implant can be implemented through minimally invasive procedures, such embodiments of the implant can pass through the interior of an access device (usually a tube having a diameter of between 5-9 mm), and then expanded inside the patient. Further, the tools for deploying the implant should also be suitable for minimally invasive procedures.

Certain embodiments disclosed herein are discussed in the context of an intervertebral implant and spinal fusion because of the applicability and usefulness in such a field. The device can be used for fusion, for example, by expanding the device to an appropriate intervertebral height and then inserting bone morphogenetic protein (BMP) or graft material. As such, various embodiments can be used to properly space adjacent vertebrae in situations where a disc has ruptured or otherwise been damaged. “Adjacent” vertebrae can include those originally separated only by a disc or those that are separated by intermediate vertebra and discs. Such embodiments can therefore tend to recreate proper disc height and spinal curvature as required in order to restore normal anatomical locations and distances. However, it is contemplated that the teachings and embodiments disclosed herein can be beneficially implemented in a variety of other operational settings, for spinal surgery and otherwise.

In addition, certain embodiments of the device can also be used to provide dynamic intervertebral support. For example, the device can be used to maintain an intervertebral height without fusion and without disc degeneration to the adjacent levels. As discussed further herein, certain components of the device can be configured to resiliently support adjacent vertebrae. In some embodiments, the device can comprise one or more components fabricated from a resilient or elastic material. The device can thus be configured to deflect within a desired range of intervertebral heights in order to provide dynamic spacing and support between adjacent vertebrae.

It is contemplated that the implant can be used as an interbody or intervertebral device. Further, the implant can be used as a tool to expand an intervertebral space or bone in order to fill the space or bone with a cement; in such cases, the implant can be removed or left in once the cement is placed. Furthermore, the implant can also be used as a tool to predilate disc space. Finally, the implant can also be introduced into the disc space anteriorly in an anterior lumbar interbody fusion (ALIF) procedure, posterior in an posterior lumbar interbody fusion (PILF) or postero lateral interbody fusion, from extreme lateral position in an extreme lateral interbody fusion procedure, and transforaminal lumbar interbody fusion (TLIF), to name a few. Although the implant is primarily described herein as being used to expand in a vertical direction, it can also be implanted to expand in a horizontal direction in order to increase stability and/or increase surface area between adjacent vertebral bodies. Therefore, it is contemplated that a number of advantages can be realized utilizing various embodiments disclosed herein. For example, as will be apparent from the disclosure, no external distraction of the spine is necessary. Further, no distraction device is required in order to install various embodiments disclosed herein. In this regard, embodiments of the implant can enable sufficient distraction of adjacent vertebra in order to properly restore disc height or to use the implant as a vertebral body replacement. Thus, normal anatomical locations, positions, and distances can be restored and preserved utilizing many of the embodiments disclosed herein.

Referring now to the figures, illustrations of certain embodiments are provided for the purpose of illustrating certain embodiments of the present inventions and not for the purpose of limiting the same.

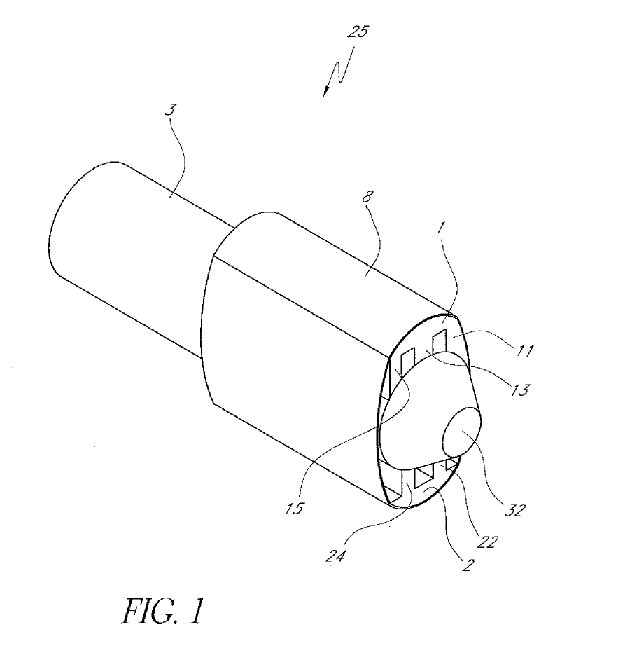

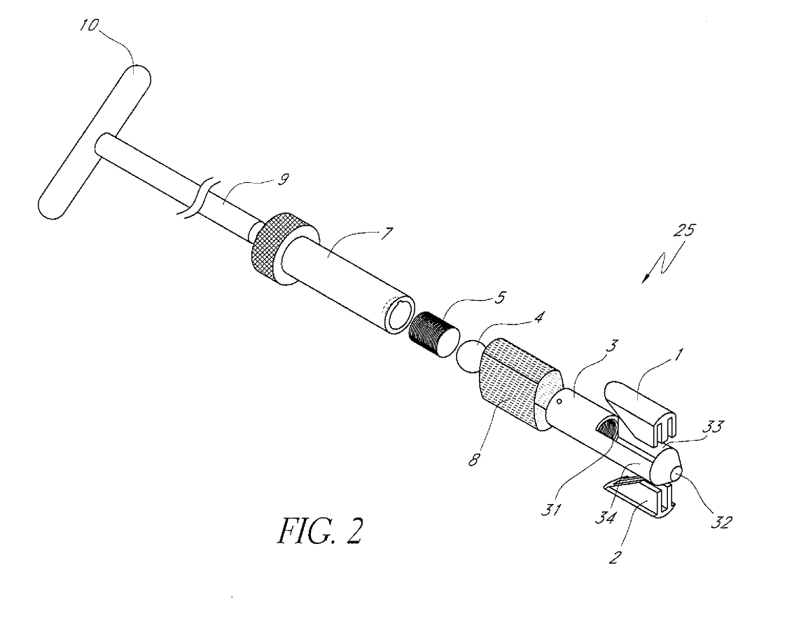

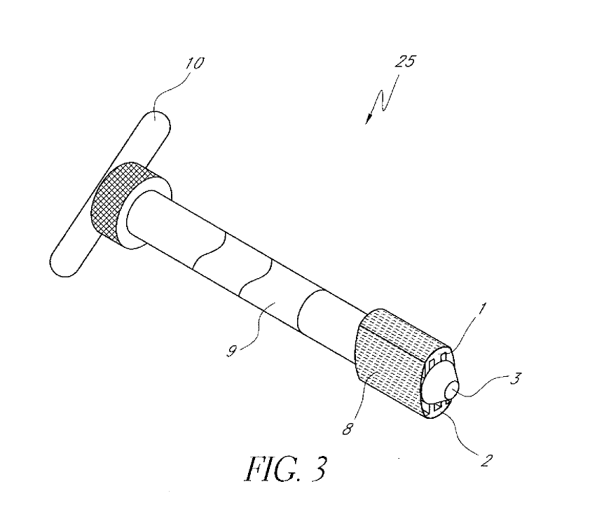

In this regard, FIGS. 1-6 illustrate an embodiment of an intervertebral implant 25 configured to be implanted using a minimally invasive procedure. Further, FIGS. 2, 3, 5 and 6 show tools that allow for manual control in minimally invasive procedure. Thus, it is contemplated that embodiments disclosed herein can pass through a cannula or other type of access device to be implanted in the spine of a patient.

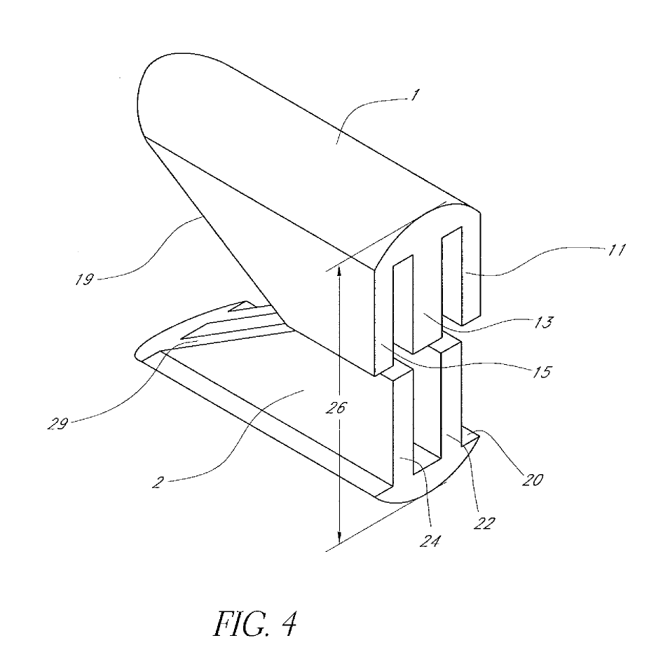

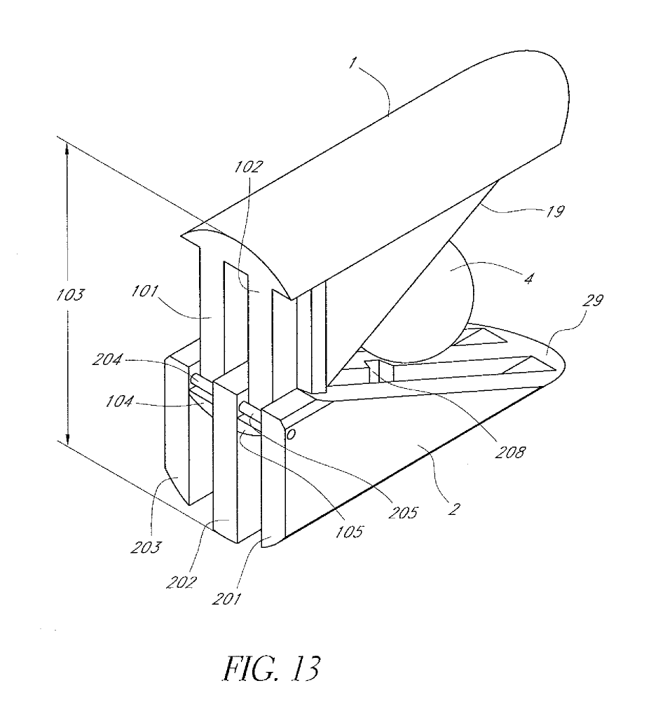

Referring now to FIGS. 1-6, an embodiment of an intervertebral implant 25 is shown. The implant 25 can comprise the first and second body portions 1, 2. The first and second body portions 1, 2 can comprise respective upper and lower external surfaces that are configured to abut against adjacent vertebrae (or intervening structure) when implanted into the spine of a patient. The separation between the external surfaces of the body portions (marked as 26 in FIG. 4 or as 103 in the FIG. 13) defines the intervertebral separation of adjacent vertebra or a implant height.

In some embodiments, the body portions 1, 2 can be configured to generally define a wedge structure. For example, the body portions 1, 2 can each define an internal contact surfaces 19, 29 (see FIG. 4). The contact surfaces 19, 29 of the respective body portions 1, 2 can each be defined at least partially by top surfaces of structural components of the body portions 1, 2. In some embodiments, the contact surfaces 19, 29 can be oriented at an incline relative to a longitudinal axis of the implant 25.

As shown in FIG. 4, the structural components of the body portions 1, 2 can each comprise one or more structures or walls that rise or extend from the outer surface or face of the body portions 1, 2. Further, the body portions 1, 2 can each define one or more slots or gaps. The embodiment illustrated in FIG. 4 illustrates the body portions 1, 2 each having a plurality of raised structures or walls 11, 22, 13, 15, 22, 24. The walls 11, 22, 13, 15, 22, 24 can comprise an angled or declining portion extending from an upstanding portion toward a base of the respective body portion. In this regard, the walls 11, 22, 13, 15, 22, 24 can each define a top surface that at least partially defines the respective contact surfaces 19, 29 of the body portion 1, 2.

For example, the first body portion 1 can comprise three walls 11, 13, 15 and a pair of slots or gaps disposed intermediate the walls 11, 13. 15. Further, the second body portion 2 can comprise a pair of walls 22, 24 in one or more slots or gaps disposed adjacent to the walls 22, 24. Accordingly, the first and second body portions 1, 2 can be interlinked by interpositioning the walls 11, 13, 15 of the first body portion 1 into the slots or gaps of the second body portion 2. As will be appreciated, such interlinking also causes the walls 22, 24 of the second body portion 2 to be disposed in the slots or gaps of the first body portion 1. In this regard, body portions 1, 2 can at least partially enter or overlay each other. Thus, the body portions 1, 2 can be configured to maximize the expansion ratio of the implant. In other words, the ratio of the height of the implant in the expanded state to the height of the implant in a collapsed state can be maximized.

Additionally, the walls 11, 22, 13, 15, 22, 24 can facilitate alignment of the first and second body portions 1, 2. Thus, the interlinking the first and second body portions 1, 2 can act as a guide for the relative motion between body portions 1, 2.

As noted above, embodiments the implant 25 can be configured to comprise body portions having one or more walls and/or one or more slots or gaps. Although it is contemplated that the walls of a body portion may be generally planar, is also contemplated that one or more of the walls can define a surface structure that facilitates alignment of the body portions relative to each other. Further, it is contemplated that the implant can incorporate an expansion limiting system. For example, the expansion limiting system can be formed such that one or more of the walls defines a surface structure that is operative to control and/or limit expansion of the body portions relative to each other.

The implant 25 can also comprise an expansion component. The expansion component can be used to cause separation between the first and second body portions 1, 2. As shown in FIGS. 2 and 5-6, in one embodiment, the expansion component can comprise a head portion 4 and a ram member 5. The head portion 4 of the expansion component can act against the first and second body portions I, 2 to control the expansion or contraction of the implant. The ram member 5 can drive the head portion 4 against the first and second body portions 1, 2.

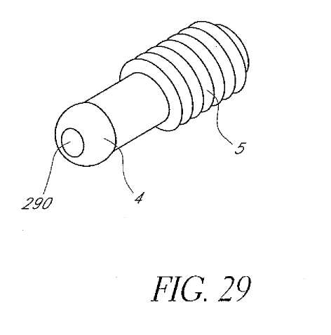

In the illustrated embodiment, the head portion 4 and the ram member 5 of the expansion component are formed separately from each other. However, in other embodiments, as illustrated in FIG. 29, the head portion and the ram member of the expansion component can be attached to each other or formed as an integral or monolithic piece.

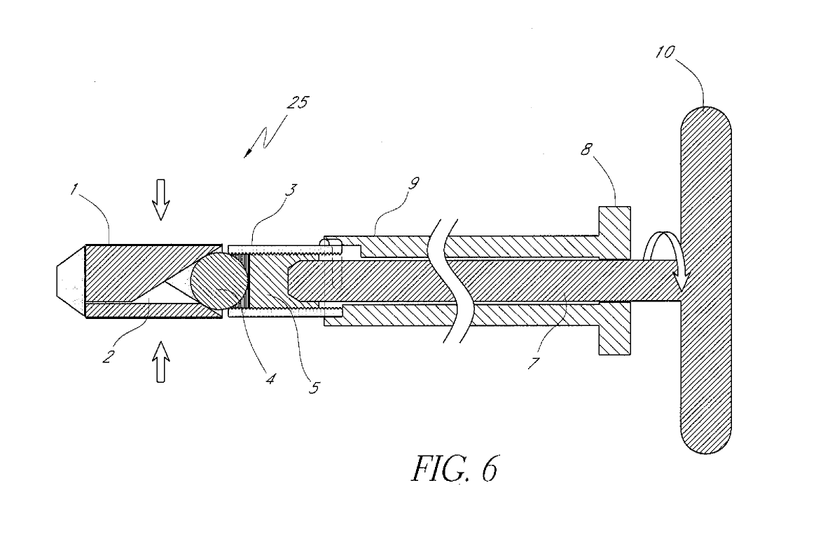

Referring to FIGS. 2 and 6, the head portion 4 of the expansion component can be formed as a spheroid. However, as discussed further above, the head portion 4 can be configured in any of a variety of geometric configurations to facilitate interaction between the expansion component and the first and second body portions 1, 2.

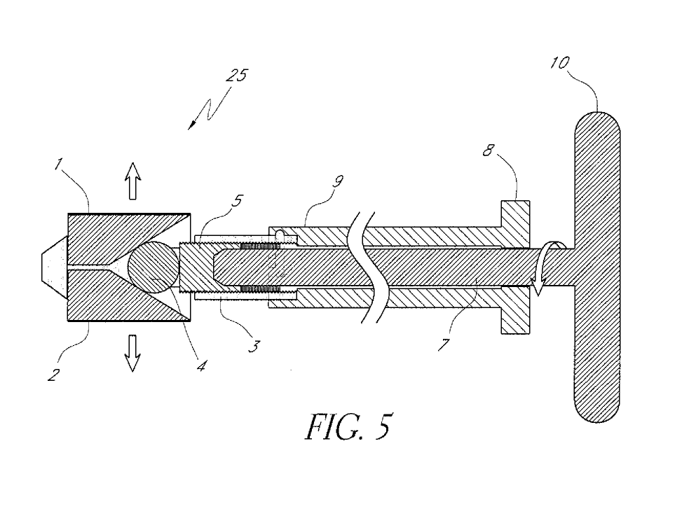

FIG. 6 illustrates the implant in a collapsed state. The head portion 4 is positioned adjacent the contact surfaces 19, 29 formed by the first and second body portions 1,2, and is in contact with the top surfaces of the walls 11, 22, 13, 15, 22, 24. The thrust of the head portion 4 against the contact surfaces 19, 29 can cause the first and second body portions 1, 2 to be separated and moved towards the expanded state shown in FIGS. 5 and 6. Accordingly, the head portion 4 can be fabricated from a non-resilient or rigid material that facilitates expansion of the implant 25 to a given intervertebral height. However, the head portion 4 can alternatively be fabricated from a resilient or elastic material. In such embodiments, a resilient head portion 4 can allow the implant 25 to be compressible. The implant 25 could then be able to provide dynamic spacing and support between adjacent vertebrae. The type of material used for the head portion 4 can therefore be chosen depending on whether the implant 25 is intended to provide support at a given height or at a range of heights (through compressibility of the implant 25). Moreover, the shape and size of the head portion 4, as well as its material properties, can be dictated by the type of therapy desired.

In some embodiments, the implant 25 can provide dynamic stabilization of adjacent vertebrae. For example, the head portion 4 can act as a shock absorber. Such shock absorption can allow the first and second body portions 1, 2 to be moved relative to each other such that the implant 25 has a degree of compressibility in an expanded state. Accordingly, the implant 25 can provide dynamic stability between the adjacent vertebrae. For example, the head portion 4 can be formed from a material with elastic properties, such as nylon or Teflon. In addition, the material should be selected so as to ensure a minimum dimensional accuracy, resilience, and stability when the implant experiences loading in the expanded state.

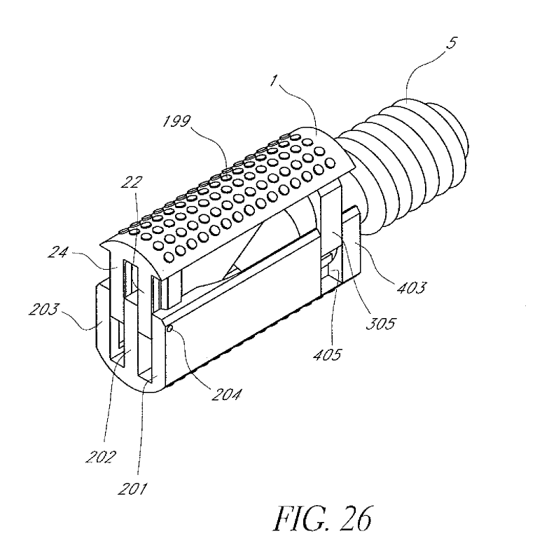

In embodiments of the implant 25 that provide dynamic stabilization of adjacent vertebrae, the first and second body portions 1, 2 can translate and/or rotate relative to each other. For example, as discussed herein, embodiments are provided in which the first and second body portions 1, 2 can be aligned relative to each other using alignment supports. These supports can generally allow vertical translation of the first and second body portions 1, 2. However, it is also contemplated that the first and second body portions 1, 2 can rotate relative to each other to provide a rocking motion between the first and second body portions 1, 2 of the implant 25. In such embodiments, the implant 25 may not use alignment supports to prevent rotational movement and maintain vertical alignment. Instead, the first and second body portions 1, 2 of the implant 25 can be can comprise one or more pins or bars, such as end caps 204, 205 shown in FIGS. 26-28. Such pins or bars can facilitate relative rotation between the first and second body portions 1, 2 of the implant 25 such that a resilient head portion 4 allows the upper surface of the first body portion 1 and the lower surface of the second body portion 2 to be angularly oriented relative to each other. In such embodiments, the first and second body portions 1, 2 of the implant 25 can advantageously expand in a vertical direction and/or rotate about a center point defined by the head portion 4. This flexibility may provide various advantages such as dynamic stabilization, customized support, and a precise implant fit that can be tailored to a given intervertebral morphology.

In addition, the intervertebral implant 25 can comprise a confinement casing 3. As illustrated in FIG. 2, the body portions 1, 2 and the expansion component can be at least partially disposed within the casing 3. In accordance with the illustrated embodiment, the casing 3 can comprise first and second ends. A channel 31 can be disposed at the first end of the casing 3. The channel 31 can be configured to at least partially receive the expansion component therein. In some embodiments, the channel 31 can comprise one or more retention structures (e.g., threads or ratchet-like mechanism). In such embodiments, the ram member 5 of the expansion component can comprise one or more retention structures (e.g., threads or ratchet-like mechanism) corresponding to the retention structures of the channel 31.

Further, the casing 3 can be configured to include a pair of sidewalls 33, 34. The sidewalls 33, 34 can extend from a channel portion of the casing 3 toward the second end of the casing 3. Finally, a lid component 32 can be formed at the second end of the casing 3. In this regard, the sidewalls 33, 34, the lid component 32, and an end face of the channel 31 can define a compartment of the casing 3.

In accordance with an embodiment, the compartment of the casing 3 can be configured such that the first and second body portions 1, 2 can be disposed therein. In this regard, the compartment and the channel 31 can be configured to at least partially house the expansion component and the first and second body portions 1, 2. Accordingly, one of the advantages of such an embodiment is that the casing 3 can restrict or limit one or more degrees of freedom of movement of the expansion component and the first and second body portions 1, 2.

For example, the casing 3 can be configured to restrict or limit relative motion of the body portions 1, 2 in a horizontal direction. Accordingly, the first and second body portions 1, 2 can be generally guided in vertical displacement during expansion or contraction of the implant. Further, the casing 3 can be configured to restrict or limit movement of the expansion component—especially if the head portion 4 is formed separately from the ram member 5. In this regard, the head portion 4 (shown as a spheroid in FIGS. 2 and 5-6) can be restricted from movement other than along a longitudinal axis of the implant. Thus, movement of the first and second body portions 1, 2 and the expansion component can be controlled or limited to selected directions such that movement of the expansion component efficiently and effectively causes expansion or contraction of the implant 25. Further, these components can be safely held together in the casing 3 in anticipation of installation and implantation, thus facilitating both handling and installation.

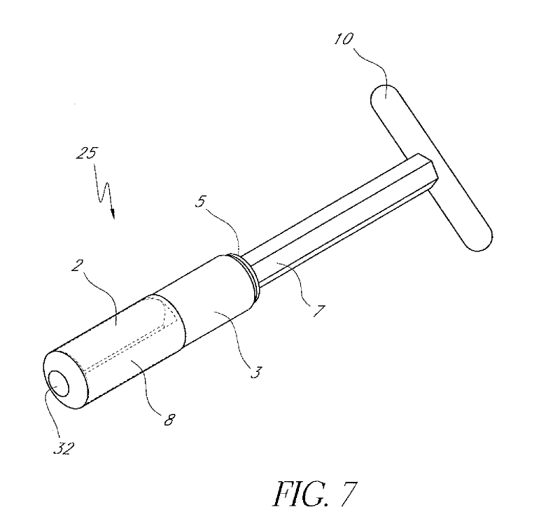

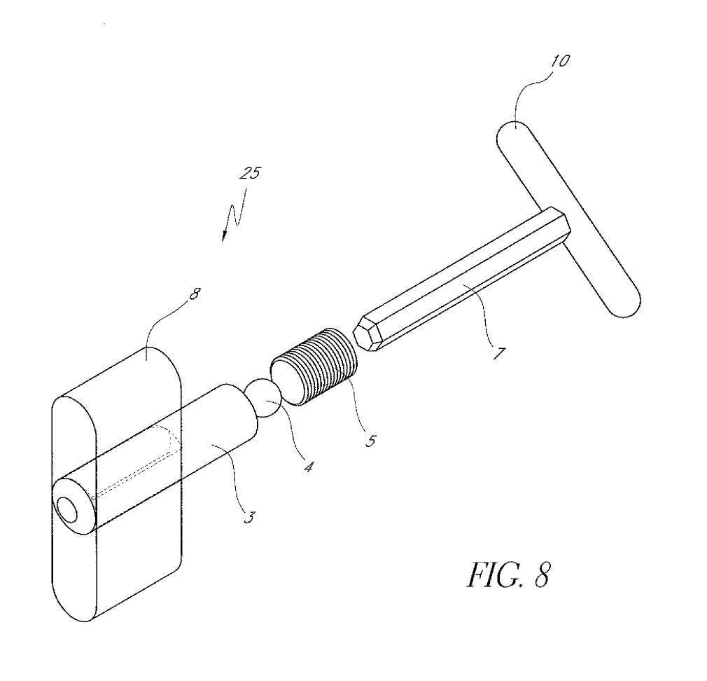

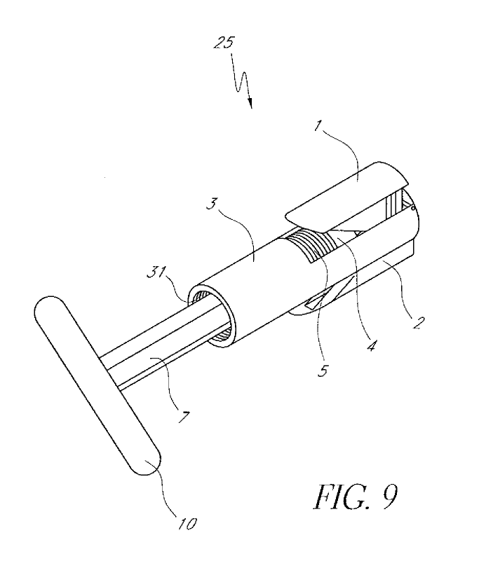

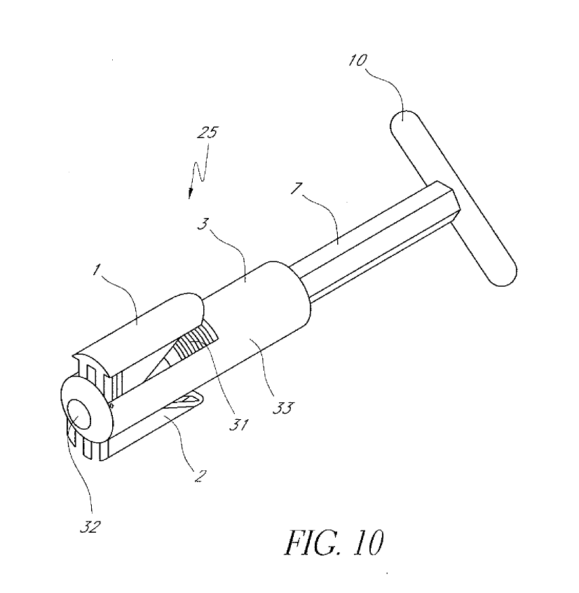

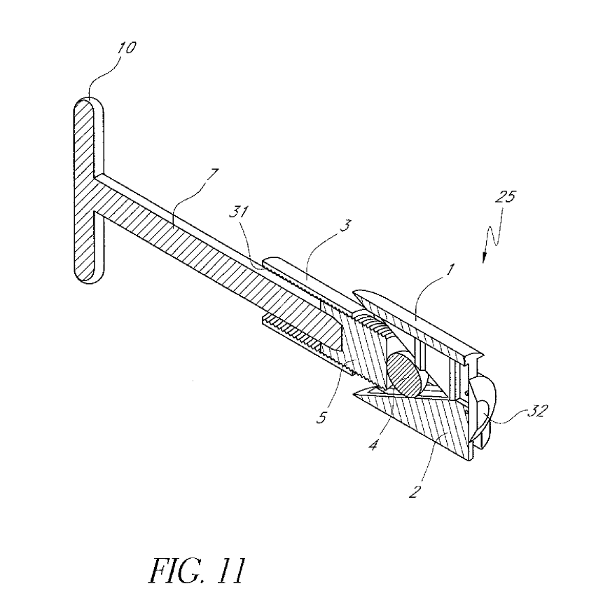

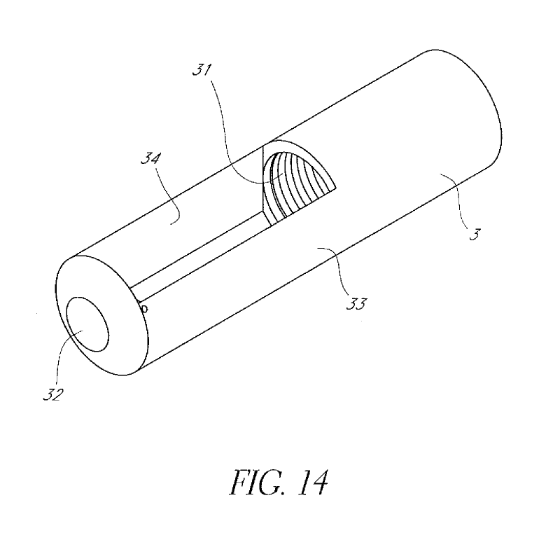

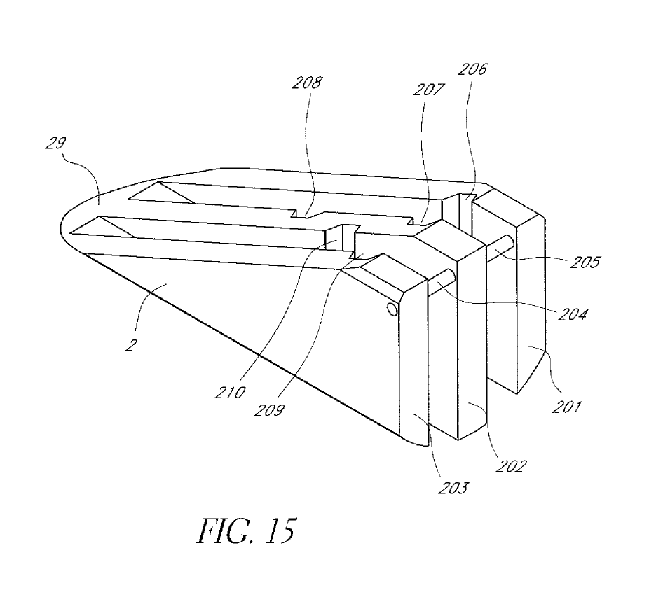

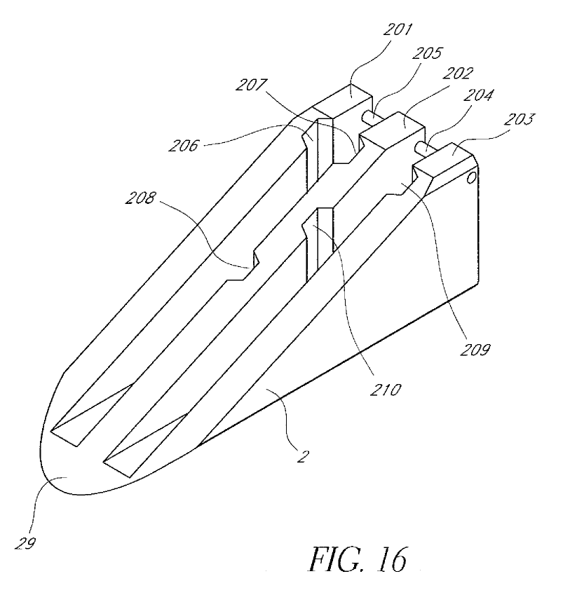

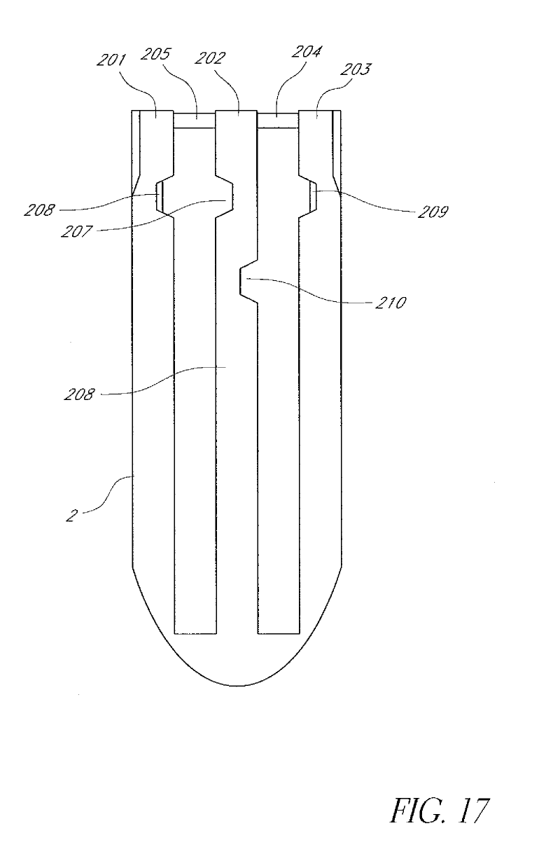

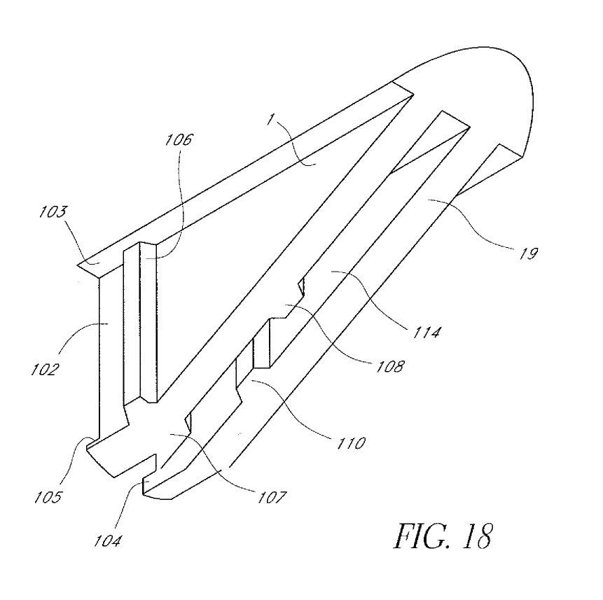

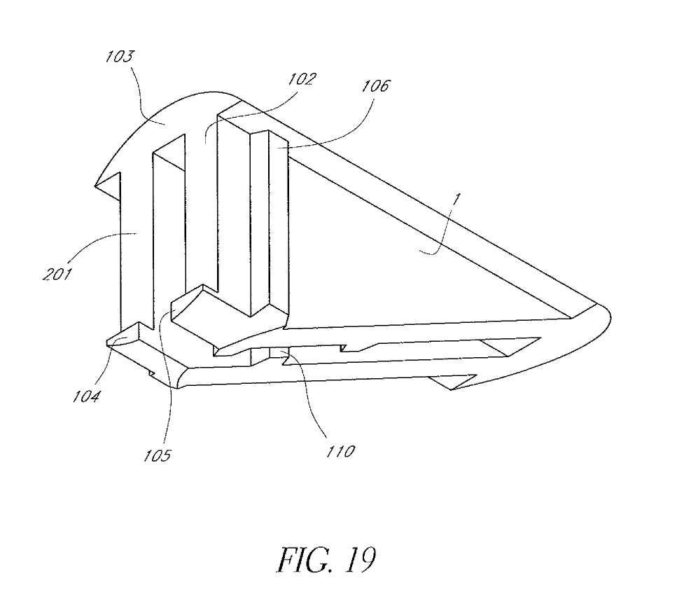

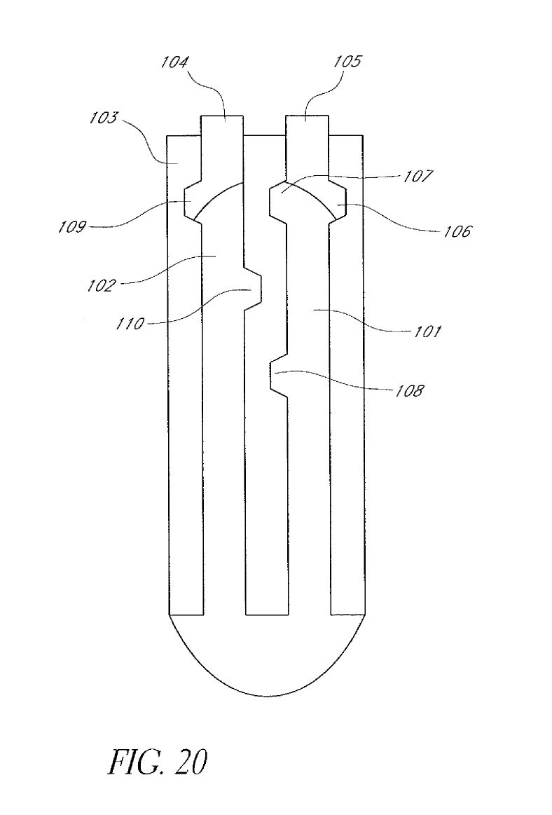

Moreover, in the embodiments shown in FIGS. 1-6 and the embodiments shown in FIGS. 7-20, the casing 3 can comprise a cylindrical shape with the compartment disposed intermediate the sidewalls 33, 34 to allow the movement of body portions. Additionally, the lid component 32 of the casing 3 can provide distal confinement to the body portions 1, 2. The channel 31 can be configured to allow introduction of deployment tools of the implant 25, such as a deployment end 7 of an expansion tool 10 shown in FIGS. 2 and 5-6.

In use, the ram member 5 is actuated by the expansion tool 10. In some embodiments, the expansion tool 10 can engage a proximal end or engagement structure of the ram member 5 in order to impart rotation to the ram member 5. As shown in FIGS. 5-6, the head portion 4 contacts the inclined contact surfaces of the first and second body portions 1, 2. In embodiments wherein the head portion 4 is formed separately from the ram member 5, the head portion 4 is pushed by an end of the ram member 5. Once installed in the casing 3, the ram member 5 and the head portion 4 can be at least partially disposed in the interior of the implant.

As noted above, the ram member 5 can comprise more retention structures. In some embodiments, the retention structures can comprise one or more threads. Further, the channel 31 can comprise corresponding threads configured to mate with the threads of the ram member 5, as shown in FIG. 5. Thus, the ram member 5 can be a threaded part (such as a threaded rod) having a first end configured to transmit axial force against the head portion 4 and a second end configured to mate with a portion of the expansion tool. The second end of the ram member 5 can comprise an engagement element configured to receive an end of the tool 7. The engagement element can comprise a geometric shape corresponding to any of a variety of geometric tooling shapes known in the art, such as an Allen hex or other types of unions. In other embodiments, the retention structures can comprise a ratchet-like mechanism between the ram member 5 and the channel 31.

One of the unique advantages provided by a threaded rain member 5 and a threaded channel 31 is that the implant can be precisely expandable with an almost endless selection of the heights. Further, the ram member 5 can also be reversible, thereby reducing the implant height, which can allows the implant to be safely removed or adjusted. In addition, threads can prevent collapse or closure of the implant.

In some embodiments, for ease of reversibility, the implant can further comprise one or more elastic recovery elements 8. The elastic recovery element 8 can extend between and interconnect the body portions 1, 2. The elastic recovery element 8 can limit and/or restrict one or more degrees of movement of the components of the implant. For example, the elastic recovery element 8 can limit the total or maximum expansion of the implant or limit axially transverse movement of the expansion component.

The elastic recovery elements 8 could be, for example, a mesh with elastic properties (e.g. a mesh with elastic material, a cut mesh, etc.) or even one or more elastic bands surrounding the body portions 1, 2. In embodiments using an elastic band, the body portions 1, 2 can further comprise an elastic groove by which the elastic band can be seated on body portions 1, 2 to prevent displacement of the elastic band from a desired position. In embodiments using a mesh, the match could be anchored to the casing 3 or the body portions 1, 2 using affixing elements, such as projections extending from the casing 3 or the body portions 1, 2, if considered necessary. Further, as discussed above, the elastic recovery elements 8 can be interconnected with areas of the body portions 1, 2 such that the elastic recovery element 8.

As it has been shown schematically in the embodiments shown in the figures, because of its structure, the implant 25 can be manipulated through a minimally invasive access device space using tools 7, 8, 9, 10. For example, the tool 10 may be manual or powered. However, it is contemplated that an Allen-type tool can be sufficient if the surgeon exercises adequate command for controlled turning of the tool. Optionally, the tool can also comprise tubular supports 8, 9 with a bayonet connection or anchor, according to known techniques.

FIGS. 7-20 illustrate other embodiment of the implant and its components shown in FIGS. 1-6. The embodiments shown in FIGS. 7-20 provide structural variations to the above-described body portions. In order to avoid repetition, components of the embodiments of the implant shown in FIGS. 7-20 that are similar to corresponding components shown and described in FIGS. 1-6 are labeled with identical numerals, and therefore will not be discussed in depth.

The body portions 1, 2 of FIGS. 7-20 can define the same general exterior shape as those of FIGS. 1-6; in other words, the body portions 1, 2 can have the general form of a wedge and can be formed having one or more raised structures or walls and one or more slots or gaps disposed adjacent to the walls.

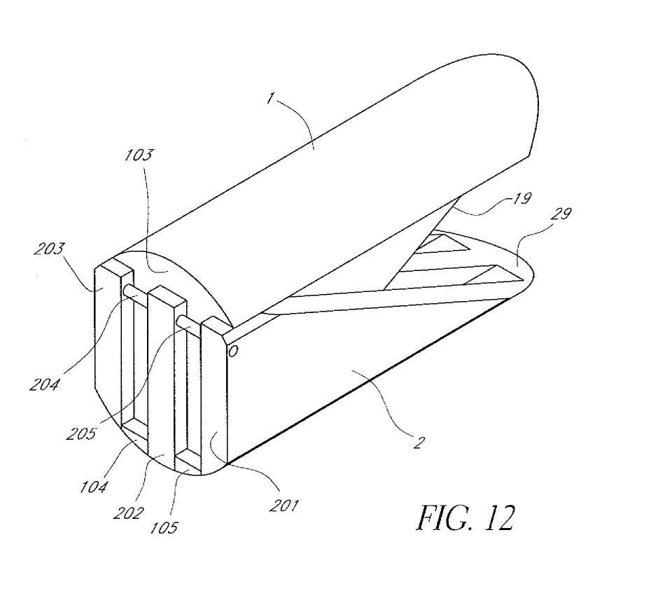

In accordance with the embodiment illustrated in FIGS. 12-13, 15 and 15-20, the first and second body portions 1, 2 of the implant can comprise one or more respective structures or walls 101, 102, 201, 202, 203 that rise or extend from the outer surface or face of the body portions 1, 2. Additionally, the side walls 101, 102, 201, 202, 203 can be configured to comprise corresponding protrusions 106, 107, 108, 109, 110 and slots 206, 207, 208, 209, 210. The protrusions 106, 107, 108, 109, 110 and slots 206, 207, 208, 209, 210 can be configured to facilitate alignment of the first and second body portions 1, 2. This can improve the vertical guidance between the body portions 1, 2, thus preventing rotational movement or axial translation between the body portions 1, 2.

Further, in accordance with some embodiments, the implant can comprise an expansion limiting system. The expansion limiting system can restrict the maximum separation or expansion between body portions 1, 2. Such a feature can be useful in some embodiments if the elastic recovery element 8 is not used.

For example, as shown in FIGS. 12-13 and 15-20, the expansion limiting system can comprise one or more tabs 104, 105 extending from the first body portion 1 that are configured to contact with end caps 204, 205 located on the second body portion 2. In the illustrated embodiment, there are two tabs 104, 105 projecting from an end of the first body portion 1 and the end caps 204, 205 have been formed using a bolt that extends through the walls 201, 202, 203. In some embodiments, the end caps 204, 205 can also serve as limits to a degree of relative rotation between the first and second body portions 1, 2. Of course, other expansion limiting systems can be formed by one of skill in the art.

Furthermore, in some embodiments, the expansion limiting system of the implant 25 can be configured to provide a retention force between the first and second body portions 1, 2 such that the first and second body portions 1, 2 are urged toward a collapsed state. In an embodiment, external or internal structures of the body portions 1, 2, such as the tabs 104, 105, can be used to implement such an elastic system of recovery. Further, the implant 25 can comprise additional components, such as a coil or a leaf spring that can be elastically deformed when the first and second body portions 1, 2 are moved to an expanded state, thus urging the first and second body portions 1, 2 toward the collapsed state. In this regard, the coil or leaf spring can exert a force tending to collapse the body portions 1, 2. However, it is contemplated that modified structures or features can be implemented to provide a retention force between the first and second body portions 1, 2.

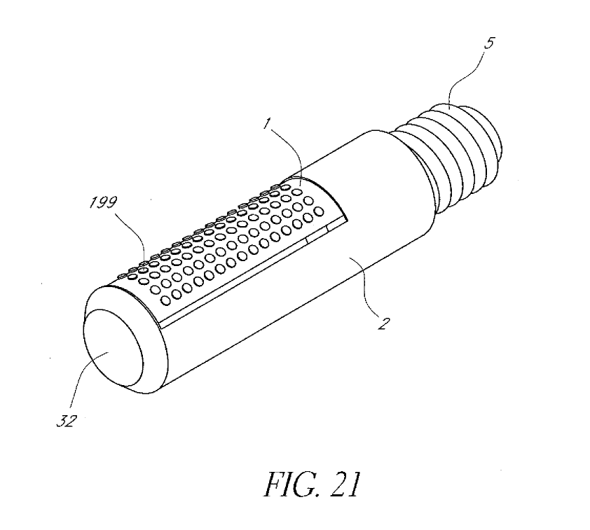

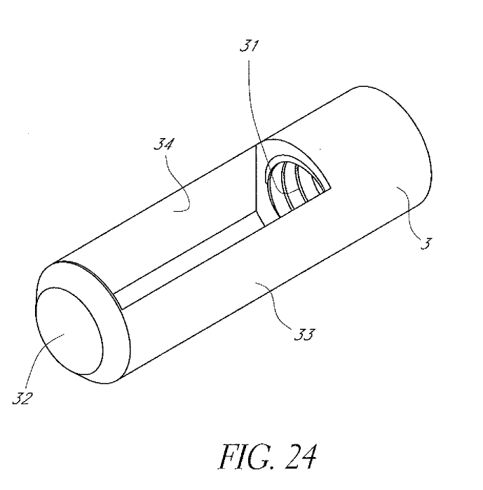

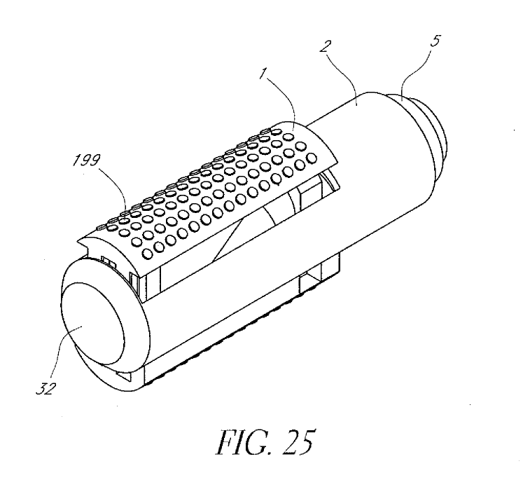

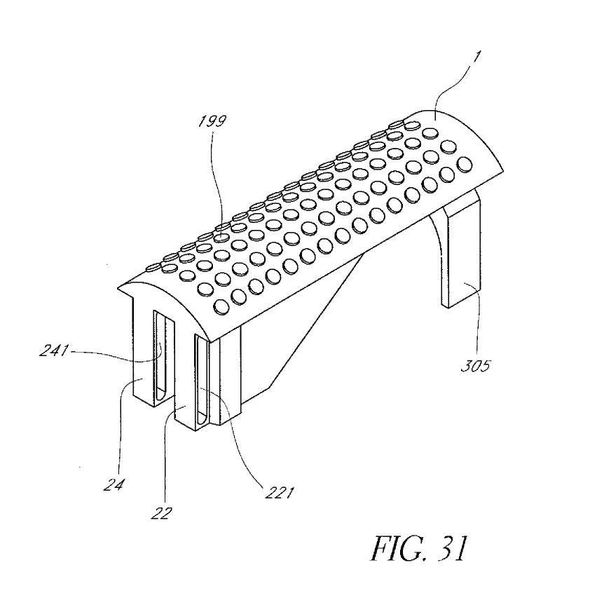

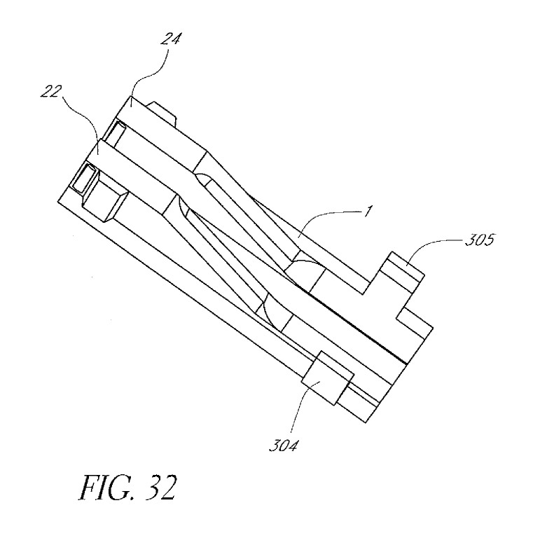

FIGS. 21-32 show yet another embodiment of an intervertebral implant. As similarly noted above with respect to FIGS. 7-20, in order to avoid repetition, components of the embodiments of the implant shown in FIGS. 21-32 that are similar to corresponding components shown and described in FIGS. 1-20 are labeled with identical numerals, and therefore will not be discussed in depth.

In accordance with the embodiments shown in FIGS. 21-32, the expansion component can comprise a head portion 4 and a ram member 5 that are interconnected. For example, the head portion 4 can be coupled to the ram member 5 via an elastic element, such as a spring. Thus, the expansion component would advantageously be handled as a single piece when one unscrews the ram member 5. In other words, some embodiments provide that the head portion 4 can be retained or coupled to the ram member 5. Accordingly, such embodiments could be implemented without the need to place, for example, an elastic recovery element as discussed above in other embodiments.

Further, in some embodiments, the head portion 4 can comprise a cavity or hollow portion 290. In this regard, in order to improve the elastic properties of the head portion 4, the cavity or hollow portion 290 can be drilled in the sphere. In this way, the head portion 4 can absorb impact made in the intervertebral space through compression into the cavity or hollow portion 290. Moreover, as noted herein, the head portion 4 can be fabricated from a resilient, compressible material.

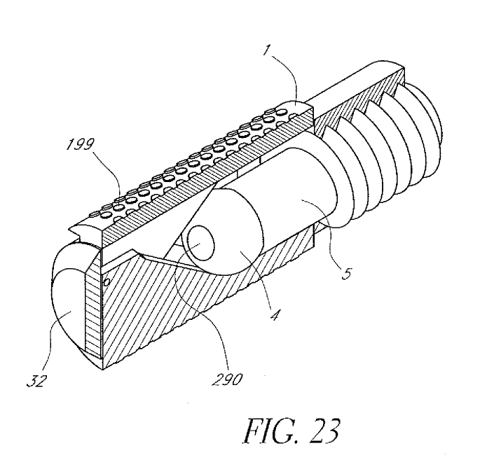

Referring now to FIGS. 23, 25, 26, and 31, some embodiments can be configured such that a surface or outer face of body portions 1, 2 comprises projections 199. The projections 199 can extend from the surface of the body portions 1, 2 for promoting osseointegration of the implant with the vertebrae. To encourage this integration, the implant can also comprise porous materials suitable for the purpose of osseointegration.

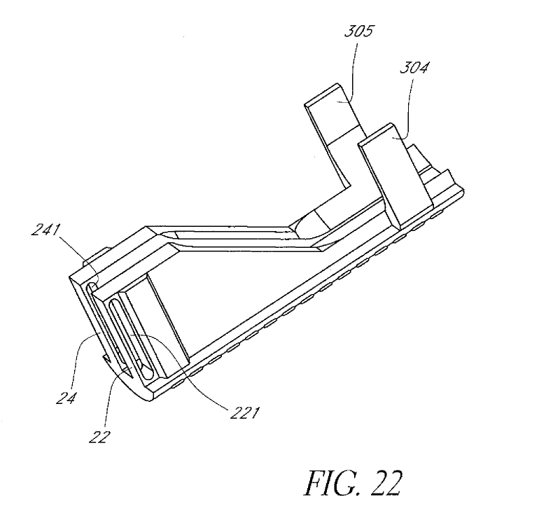

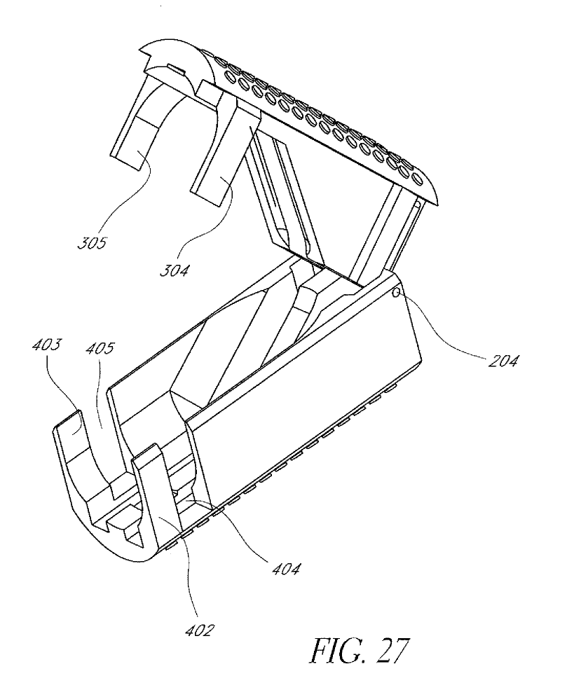

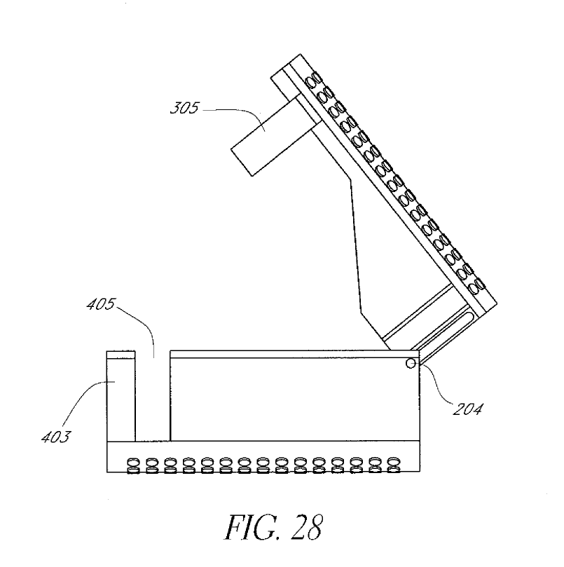

With regard to FIGS. 22, 25-28, and 30-32, some embodiments can be configured such that the body portions 1, 2 comprise alignment supports 304, 305, 402, 403, 404, 405. The alignment supports 304, 305, 402, 403, 404, 405 can extend from the body portions 1, 2 and be configured to prevent rotation, and/or torsion between the body portions 1, 2 during relative vertical movement between the body portions 1, 2. FIGS. 27 and 28 illustrate orientations of the body portions 1, 2 in which the body portions 1, 2 are rotated relative to each other. In some embodiments, such rotation may not be prevented by the implementation of an expansion limiting system alone. In the illustrated embodiment, the expansion limiting system can comprise the interaction of the slots 221, 241 with the end caps 204, 205. As shown, the end caps 204, 205 have been formed using a bolt. However, in some embodiments, the expansion limiting system can be used in conjunction with the alignment supports 304, 305, 402, 403, 404, 405 to prevent rotation of the body portions 1, 2 relative to each other during relative vertical movement thereof. Furthermore, in embodiments that do not include the alignment supports 304, 305, 402, 403, 404, 405 or corresponding protrusions 106, 107, 108, 109, 110 and slots 206, 207. 208, 209, 210 discussed above, the end caps 204, 205 can facilitate a degree of relative rotation between the first and second body portions 1, 2.

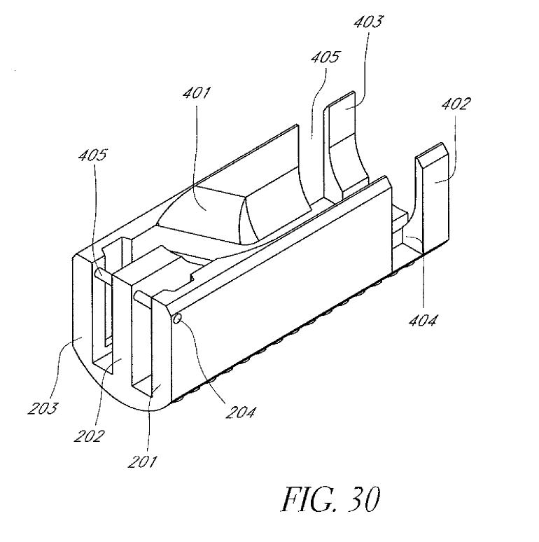

Similarly, the body portions 1, 2 can comprise a rounded edge 401, as shown in FIG. 30. Thus, in such an embodiment, the head portion 4 of the expansion component can be seated against the rounded edge 401 during longitudinal movement of the expansion member. Such an embodiment advantageously provides a greater area of contact with the body portions 1, 2, and distributes a load more evenly through the components of the implant.

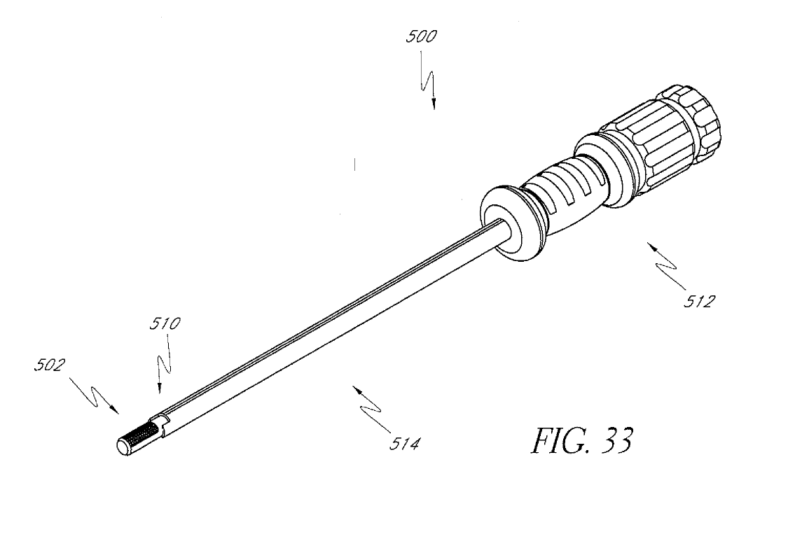

In accordance with another embodiment, FIG. 33 illustrates a perspective view an installation tool 500 and an intervertebral implant 502 seated in an engagement portion 510 of the tool 500. As illustrated, the installation tool 500 can comprise a handle portion 512 and a deployment portion 514. The installation tool 500 can be used to place and deploy the implant 502 during a medical procedure. As discussed herein, embodiments of the installation tool 500 and the implant 502 provide significant advantages over prior art installation tools and implants.

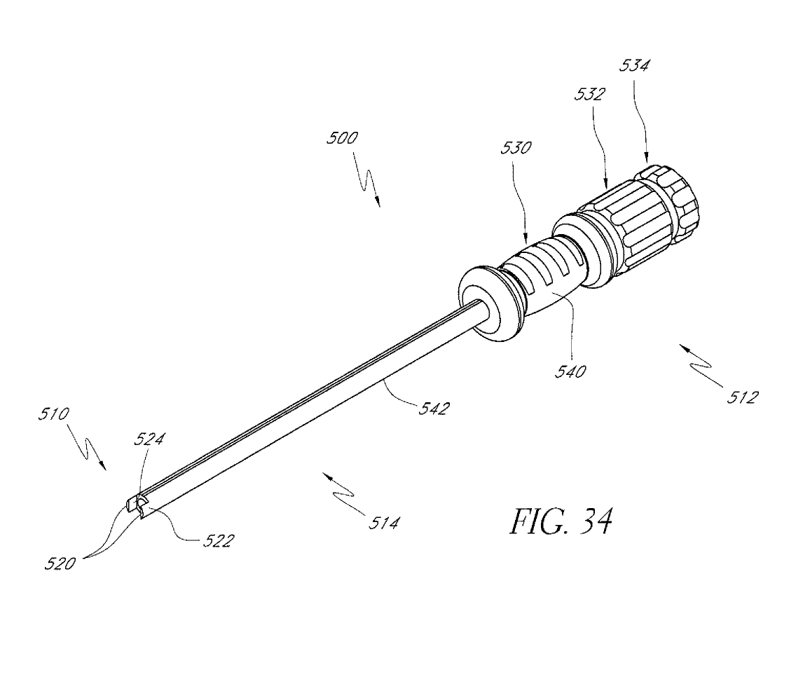

FIGS. 34-37 illustrate the installation tool 500 in greater detail. In the illustrated embodiment, the handle portion 512 can be configured to facilitate placement and operation of the implant 502, such as controlling the expansion or contraction of the implant. As shown in FIG. 34, the engagement portion 510 of the installation tool 500 can comprise one or more protrusions 520 extending distally from a distal end 522 of the deployment portion 514. In other words, the engagement portion 510 can extend distally from the distal and 522 of the deployment portion 514. The one or more protrusions 520 of the engagement portion 510, as well as other structures of the installation to 500, can be used to engage and retain the implant 502 on the installation tool 500 during placement of the implant 502. Further, in some embodiments, the one or more protrusions 520 and other structures can also enable a surgeon to deploy, remove, expand, and/or contract the implant 502.

Referring now to FIG. 34, in some embodiments, the protrusions 520 of the engagement portion 510 can be configured to extend generally parallel relative to a longitudinal axis of the deployment portion 514. Further, the protrusions 520 can comprise surfaces 524 that face each other. In some embodiments, the surfaces 524 can be flat. Additionally, the surfaces 524 can face each other and be generally parallel relative to each other. In accordance with at least one embodiment, the surfaces 524 can serve to engage a portion of the implant 502 during placement and operation of the implant 502. For example, the surfaces 524 can maintain a rotational orientation of the implant 502 relative to the longitudinal axis of the deployment portion 514.

In other words, in some embodiments, the engagement portion 510 can be configured to restrain at least one degree of movement of the implant 502 relative to the installation tool 500. However, as will be appreciated, the engagement portion 510 can be configured with a single protrusion having a uniquely shaped engagement structure that can mate with a corresponding engagement structure of the implant 502. For example, the protrusion can have any of a variety of shapes, such as a star or flat shape, a square shape, or other polygonal shapes. In this regard, the implant 502 can also comprise a structure corresponding to the shape of the protrusion; the structure can be a recess or other protrusion that facilitates mating engagement between the implant 502 and the protrusion of the engagement portion 510.

Referring still to FIG. 34, the handle portion 512 of the tool 500 can comprise several components. For example, in the illustrated embodiment, the handle portion 512 comprises a handle member 530, a first rotating member 532, and a second rotating member 534. As shown at FIGS. 34-36, the handle member 530, the first rotating member 532, and the second rotating member 534 get each comprise a distal elongate component that can form a part of the deployment portion 514 and a proximal component that can form a part of the handle portion 512.

For example, as illustrated in FIG. 34, the handle member 530 includes a gripping component 540 and an elongate tubular component 542. The gripping component 540 is coupled to the tubular component 542 such that the tubular component 542 does not rotate with respect to the gripping component 540. However, as will be discussed further here in, some embodiments of the tool 500 provide that at least one of the first and second rotating members 532, 534 rotate with respect to a longitudinal axis of the handle member 530. Accordingly, a surgeon can grasp the gripping component 540 in one hand and use the other hand to rotate one of the first and second rotating members 532, 534. In this manner, the surgeon can control implant 502.

As discussed above, the deployment portion 514 can comprise the engagement portion 510. In some embodiments, the handle member 530 can comprise the engagement portion 510. More specifically, the tubular component 542 can comprise the engagement portion 510. As such, in some embodiments the surgeon can grasp and use the gripping portion 540 two per event rotation of the engagement portion 510. Thus, the surgeon can ensure that the implant 502 maintains a desired rotational alignment during placement and deployment at the deployment site.

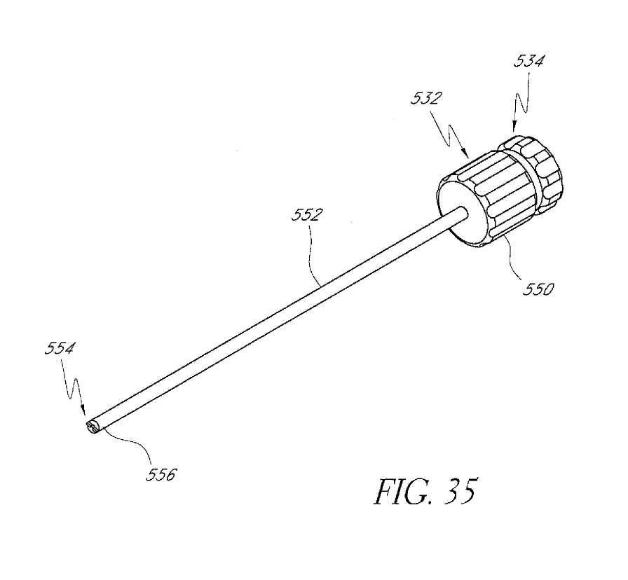

Referring now to FIG. 35, the first and second rotating members 532, 534 are shown separate from the handle member 530. The first rotating member 532 can comprise a first knob in 550 at an actuation component 552. In some embodiments, the first knob 550 is coupled to the actuation component 552 to prevent relative movement between the first knob 550 at the actuation component 552. Further, the actuation component 552 can be configured to pass through a part of the tubular component 542 of the handle member 530. For example, the actuation component 552 can extend through a bore of the tubular component 542. The actuation component 552 can be rotatable with respect to a bore or opening of the tubular component 542. Further, the actuation component 552 can comprise a generally cylindrical outer profile.

Additionally, in the illustrated embodiment actuation component 552 is configured as an elongate tubular member having a rotational connector 554 disposed at a distal end 556 thereof. In this regard, the rotational connector 554 can be configured to interact with the implant 502 so as to control one or more operations of the implant 502. For example, when the implant 502 is engaged with the installation tool 500, the rotational connector 554 can engage the ram member of the implant 502 to move the implant 502 to an expanded or contracted configuration.

In some embodiments, the rotational connector 554 can comprise one or more protrusion that engage the ram member of the implant 502. For example, in the illustrated embodiment, the rotational connector 554 can comprise one or more protrusion that extend distally from the actuation component 552. In particular, the rotational connector 554 can comprise a pair of generally rectangular protrusions that extend transversely relative to a longitudinal axis of the first rotating member 532.

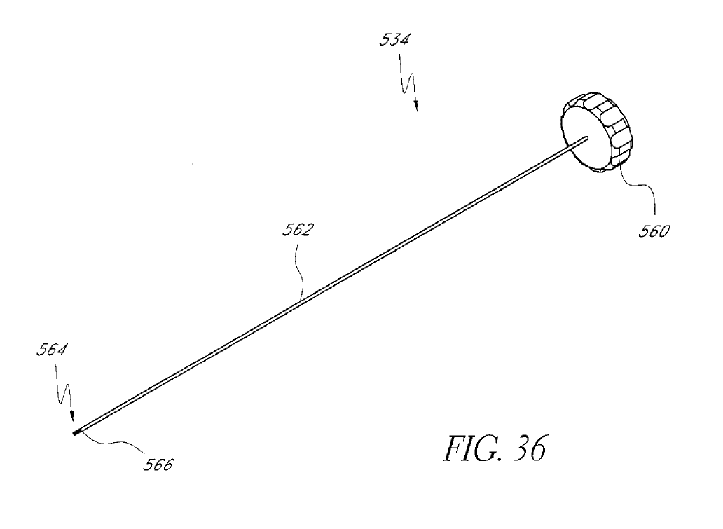

Additionally, FIG. 36 illustrates an embodiment of the second rotating member 534. The second rotating member 534 can comprise a second knob 560 and a retention component 562. Further, the retention component 562 can comprise a fastening portion 564 disposed at a distal and 566 thereof. The second knob 560 can be coupled to the retention component 562 to prevent relative rotational therebetween. Accordingly, in an embodiment, rotation of the second knob 560 can cause rotation of the fastening portion 564. In some embodiments, the fastening portion 564 can comprise one or more threads that are configured to engage or corresponding threads of the implant 502. Further, the retention component 562 can be configured to extend within a bore or opening of the actuation component 552 of the first rotating member 532. The retention component 562 can be rotatable with respect to the bore or opening of the actuation component 552. Further, the retention component 562 can comprise a generally cylindrical outer profile.

In this regard, the second rotating member 534 can be configured such that the fastening portion 564 extends distally beyond at least a portion of the distal end 556 of the actuation component 552. Further, both the fastening portion 564 of the second rotating member 534 and the rotational connector 554 of the first rotating member 532 can be configured to extend distally beyond at least a portion of the distal end 522 of the tubular component 542.

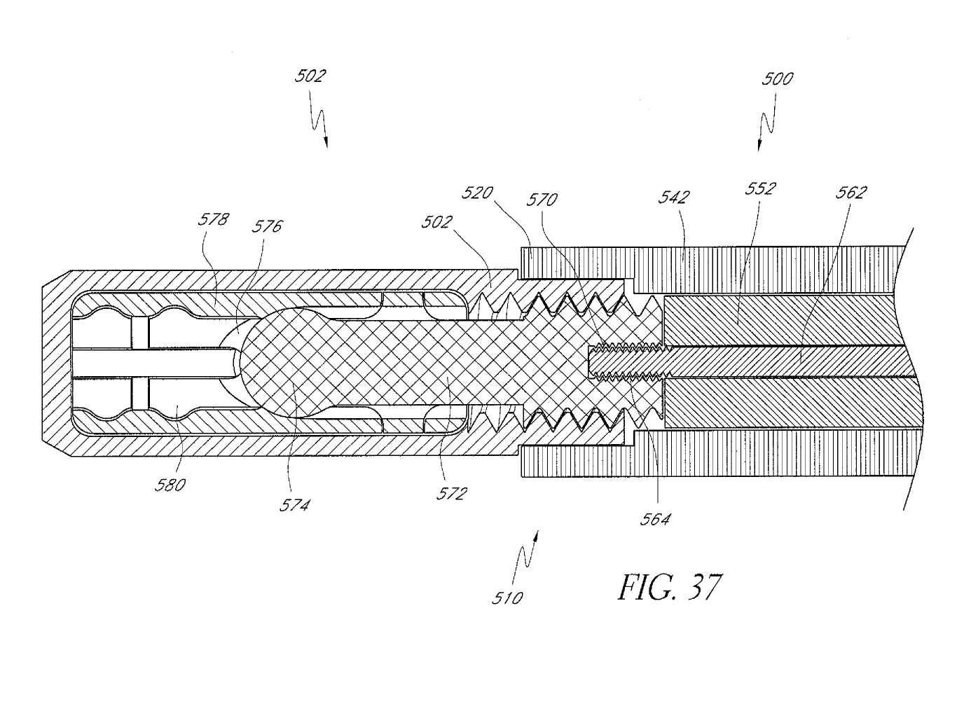

For example, as illustrated in FIG. 37, the retention component 562 can extend within the actuation component 552, which can likewise extend within the tubular component 542. The retention component 562 can rotate relative to both the actuation component 552 and the tubular component 542 in order to engage a recess 570 of the implant 502. In some embodiments, the recess 570 can be threaded. Thus, in use, a casing 504 of the implant 502 can be positioned with in the engagement portion 510 of the tool 500 and the second knob 562 can be rotated to cause the retention component 562 to engage the recess 570 of the implant 502 in order to couple the implant 502 with the tool 500. In other embodiments, the implant 502 can comprise a recess that is not threaded, but that comprises one or more protrusions or detents that allow the retention component 562 to engage the implant 502.

The coupling between the implant 502 and the tool 500 is facilitated at least in part due to the engagement between the one or more protrusions 520 of the engagement portion 510 that serve to restrict the relative rotation between the casing 504 of the implant 502 and the tool 500. Further, the engagement between the retention component 562 and the implant 502 can serve to draw the casing 504 of the implant 502 into the engagement portion 510 of the tool 500. In this manner, the second rotating member 534 can facilitate retention between the tool 500 and the implant 502. Embodiments of this system provide various benefits and advantages such as improved engagement between the implant 502 and the tool 500, as well as precise implant actuation and improved deployment control for the surgeon.

Once the implant 502 is engaged by the retention component 562, the actuation component 552 can also be used to engage a portion of the implant 502. For example, the actuation component 552 can be configured to engage a ram member 572 of the implant 502. The first knob 550 can be rotated to cause the actuation component 552 to rotate the ram member 572 of the implant 502. As the ram member 572 rotates, a head 574 of the ram member 572 can be urged against at least one inclined surface 576 of the implant 502, which causes first and second portions 578, 580 of the implant 502 to move relative to each other to create a change in implant height, such as by moving from an expanded to a contracted configuration, or vice versa. In this manner, the first rotating member 532 can facilitate expansion or contraction of the implant 502.

Additionally, it is contemplated that in some embodiments, the retention component 562 and the actuation component 552 can be axially movable relative to the tubular component 542. In this manner, as the ram member 572 is rotated, which causes the ram member 572 to be drawn into the casing 504 of the implant 502, the retention component 562 and the actuation component 552 can move axially with the proximal end of the ram member 572 to maintain engagement therebetween. In such an embodiment, it is also contemplated that the proximal end of the casing 504 can abut one or more shoulders or stops formed in the engagement portion 510 of the tool 500 during expansion of the implant 502. As such, axial movement of the retention component 562 and the actuation component 552 can take place while the proximal end of the implant 502 abuts the shoulders or stops of the engagement portion 510. Such an embodiment can ensure that the casing 504 is fully engaged with the engagement portion 510 during placement and deployment. However, in other embodiments, it is contemplated that the engagement portion 510 of the tool 500 can be configured to allow the proximal end of the casing 504 to be drawn further thereinto without creating interference against the proximal end of the casing 504 during expansion of the implant.

One of the unique advantages of the illustrated embodiment of the tool 500 is that in use, both the tubular component 542 and the actuation component 552 can operate to restrict rotational movement of the casing 504 of the implant 502 while the retention component 562 is rotated to either engage or disengage with the rain member 572 of the implant 502. Thus, even after placement of the implant 502, the torque required to disengage the retention component 562 from the recess 570 of the ram member 572 can be generally negated by applying a countervailing torque to the tubular component 542 and the actuation component 552. Thus, once in a deployed state or final position, the placement of the implant 502 need not be disturbed during disengagement of the tool 500. Similar advantages are present with regard to relative rotation between the actuation component 552 and the tubular component 542 in order to move the implant 502 to an expanded or a contracted configuration. Accordingly, the tool 500 provides the surgeon with a superior degree of control in placing and deploying the implant 502.

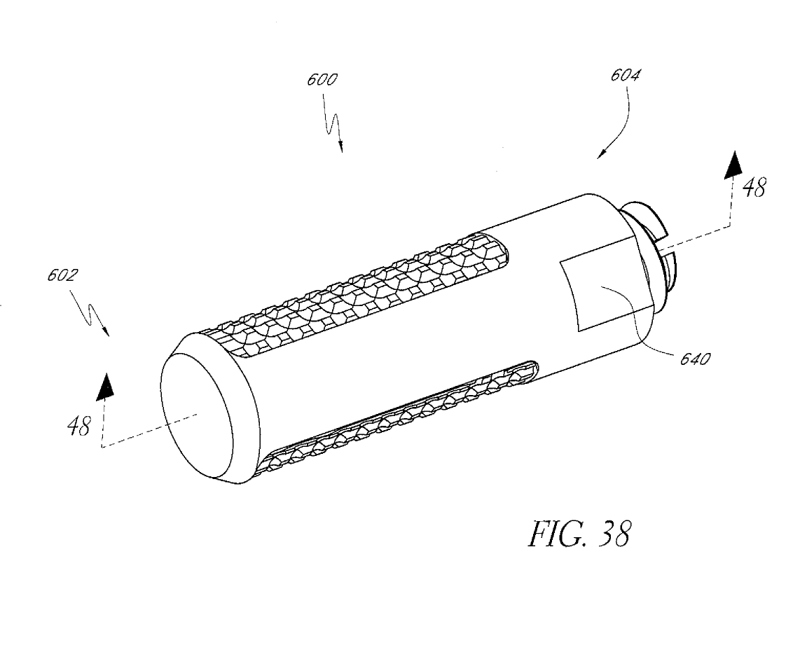

With regard now to FIG. 38, another embodiment of an intervertebral implant 600 illustrated. In FIG. 38, the implant 600 shown in a collapsed or undeployed configuration. As such, the implant 600 shown in FIG. 38 defines a minimal passing profile that allows the implant 600 to be placed at a desired intervertebral position for deployment. As discussed herein, the implant 600 can be maneuvered and operated using an installation tool, such as the tool 500 discussed above. The implant 600 can comprise a distal and 602 and a proximal end 604. The proximal and 604 can be engaged by the installation tool in order to place and cause the expansion or contraction of the implant 600.

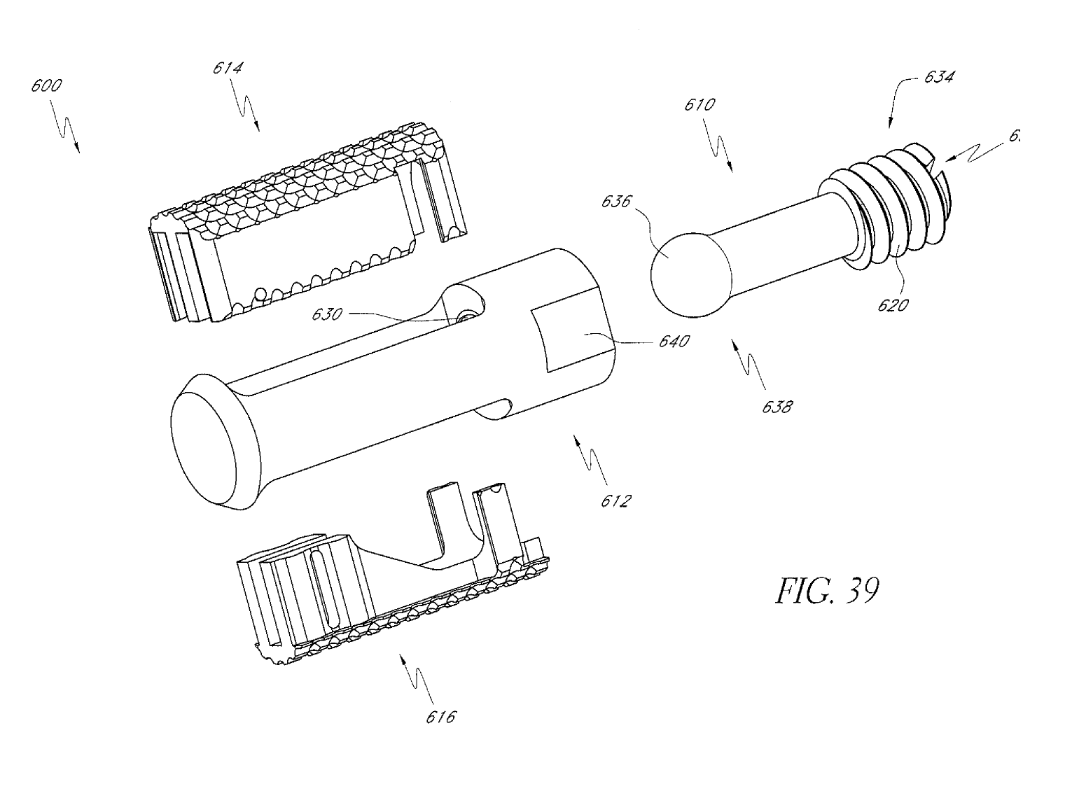

As shown in FIG. 39, the illustrated embodiment of the implant 600 can comprise several components. The implant 600 can comprise an expansion component 610, a casing 612, a first body portion 614, and a second body portion 616. The expansion component 610 can have a ram member and a head. In at least one embodiment, the operation of the implant 600 is similar to the operation of the implants discussed above.

For example, in the embodiment illustrated in FIGS. 38-48, the casing 612 of the implant 600 is configured to receive the expansion component 610. Further, a threaded portion or ram member 620 of the expansion component 610 can threadably engage internal threads of an inner bore 630 of the casing 612. In this regard, the expansion component 610 can rotate relative to the casing 612 by application of a rotational force to the expansion component 610. In order to facilitate transfer of a rotational force to the expansion component 610, the expansion component 610 can comprise one or more engagement structures 632 disposed at a proximal end 634 of the expansion component 610. Accordingly, as illustrated in the exemplary embodiment of FIG. 37, a portion of an installation tool can engage one or more engagement structures 632 of the expansion component 610 in order to transfer a rotational force to the expansion component 610 via the ram member 620.

Further, the rotational movement of the expansion component 610 can cause the expansion component 610 to move axially relative to the casing 612. As a result, a head 636 of the expansion component 610 disposed at a distal end the 638 of the expansion component 610 can be urged against internal structures are surfaced as of the first and second body portion 614, 616. In this regard, the first and second body portion 614, 616 can be separated by the rotational movement of the expansion component 610, thus causing the implant 600 to expand.

Furthermore, in some embodiments, the rotational movement of the expansion component 610 can be isolated from the casing 612 by restricting rotational movement of the casing 612. In this regard, the casing 612 can comprise one or more structures that can be engaged by the tool 500 in order to retain the casing 612 in a given rotational position as the expansion component 610 is rotated. In other words, as discussed herein, the tool 500 can be configured to engage multiple portions of the implant 600 in order to selectively rotate portions of the implant 600 relative to each other or portions of the implant 600 relative to portions of the tool 500.

In the illustrated embodiment of FIG. 39, the easing 612 can comprise one or more engagement surfaces 640. As shown, the casing 612 can comprise a pair of engagement surfaces 640 that are disposed on opposite sides of the casing 612. The illustrated embodiment indicates that the casing 612 can be configured to define a generally cylindrical configuration and that the engagement surfaces 640 can be formed as generally flat sections disposed along the perimeter of the casing 612. In this embodiment, the engagement surfaces 640 can be configured to mate with the surfaces 524 of the protrusions 520 of the engagement portion 510 of the tool 500.

In use, when the surfaces 524 of the engagement portion 510 are mated with the engagement surfaces 640 of the casing 612, relative rotational movement is restricted between the elongate tubular component 542 of the tool 500 and the casing 612 of the implant 600. Thus, other portions of the tool 500 can actuate other portions of the implant 600. For example, the actuation component 552 can rotate the expansion component 610 while the rotational movement of the casing 612 is fixed. Accordingly, the implant 600 can be actuated by the tool 500 to control the height and/or expansion of the implant 600.

As discussed above, the first and second rotating members 532, 534 of the installation tool 500 can be used to interact with the implant 600. One of the unique advantages provided by the embodiments of the implant 600 at the tool 500 is that relative motion between the tool 500 at the implant 600 can be controlled using the tool 500. For example, as noted above, the tubular component 542 of the handle member 530 can engage with the casing 612 of the implant 600, thereby preventing rotation of the implant 600 relative to the handle member 530.

Additionally, the fastening portion 564 of the second rotating member 534 can engage the recess 570 of the expansion component 610 of the implant 600 in order to couple the implant 600 to the tool 500. Further, in some embodiments, the tool 500 can be configured to prevent rotation of the expansion component 610 as the fastening portion 564 of the second rotating member 534 is coupled to the recess 570 of the implant 600. In other embodiments, the implant 600 can comprise a recess that is not threaded, but that comprises one or more protrusions or detents that allow the fastening portion 564 to engage the implant 600. Thus, the fastening portion 564 can be axially urged distally into the recess of the implant 600 to become engaged therewith. In order to disengage the fastening portion 564 from the implant 600 in such an embodiment, the actuation component 552 can abut the proximal end of the implant 600 to prevent proximal movement of the implant 600 as the fastening portion 564 is proximally removed from the recess of the implant. In such embodiments, the tool 500 can be coupled to or disengaged from the implant 600 without causing torque or axial movement to be passed to the implant 600 once in a desired deployment position.

For example, the rotational connector 554 of the first rotating member 532 can engage the expansion component 610 of the implant 600 in order to prevent rotation of the expansion component 610 relative to the first rotating member 532. Thus, in order to couple the fastening portion 564 to the recess 570, the surgeon can position the implant 600 against the engagement portion 510, engage the rotational connector 554 with the expansion component 610, grasp the first knob 550, and rotate the second knob 560 relative to the first knob 550 in a given direction. Similarly, to detach the fastening portion 564 from the recess 570, the surgeon can rotate the second knob 560 relative to the first knob 550 in a direction opposite to the given direction.

Finally, the interaction of the tool 500 in the implant 600 is also unique in that the first rotating member 532 can be used to actuate expansion or contraction of the implant 600 through rotation while the tubular component 540 to engage as the casing 612 to prevent the implant 600 from rotating with the rotation of the first rotating member 532. In other words, rotation of the casing 612 can be prevented during rotation of the expansion component 610. In use, the surgeon may rotate the first and second rotating members 532, 534 relative to the handle member 530 in order to rotate the expansion component 610 relative to the casing 612. Thus, the expansion component 610 can cause the first and second body portions 614, 616 of the implant 600 to move relative to each other. In this manner, the tool 500 enables the surgeon to carefully control expansion and contraction of the implant 600. The surgeon can isolate rotational movement of portions of the tool 500 relative to each other, portions of the implant 600 relative to each other, and portions of the tool 500 relative to portions of the implant 600.

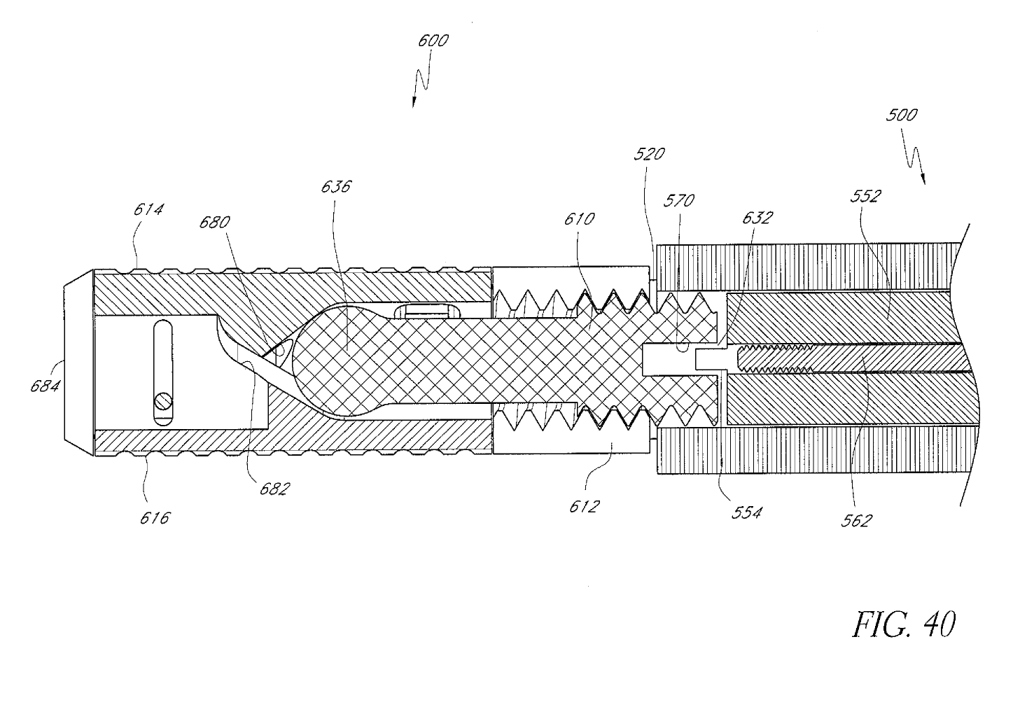

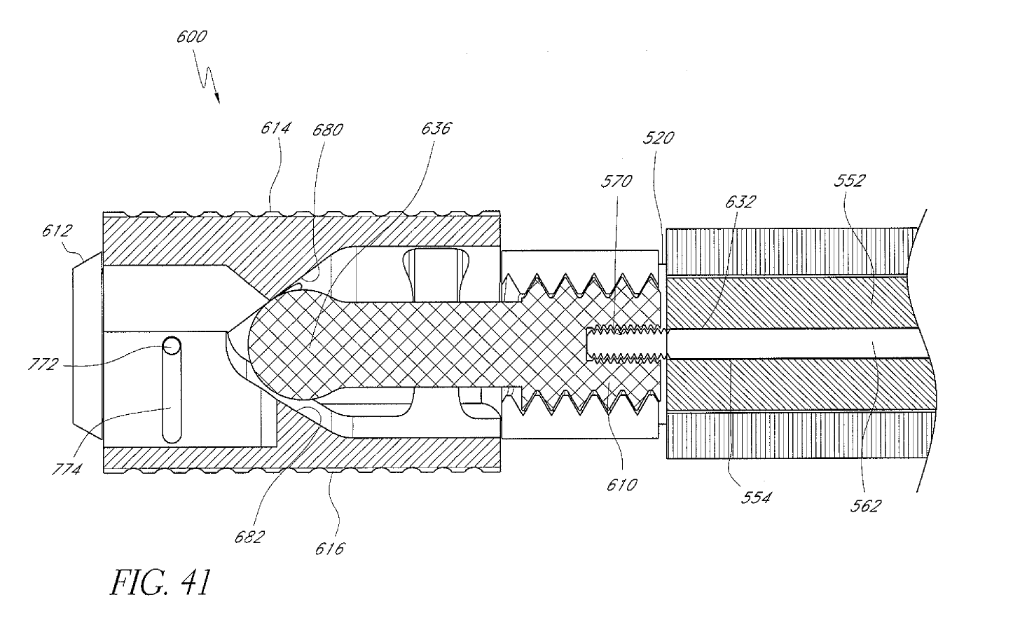

FIG. 40 illustrates a cross-sectional side view of the implant 600 in a collapsed configuration or state. As illustrated, the installation tool 500 can be coupled to the implant 600 in order to position and to deploy the implant 600. In this regard, as previously noted with respect to FIG. 37, the retention component 562 can be engaged with the recess 570 of the implant 600. Although FIGS. 40 and 41 indicate that the recess 570 is threaded, the recess 570 can comprise threads, protrusions, or detents that facilitate engagement between the retention component 562 and the recess 570. Further, the rotational connector 554 of the actuation component 552 can be engaged with the engagement structures 632 of the expansion component 610 in order to rotate the expansion component 610.

As shown FIG. 40, the first and second body portions 614, 616 can comprise respective contact surfaces 680, 682. The contact surfaces 680, 682 can be generally transversely oriented with respect to a longitudinal axis of the implant 600. In some embodiments, the contact surfaces 680, 682 can be inclined with respect to the longitudinal axis. For example, as shown in FIG. 40, the contact surfaces 680, 682 can be generally planar surfaces configured to contact the head 636 of the expansion component 610. As discussed similarly above with respect to other embodiments of the implant, as the head 636 is urged distally or toward a distal end 684 of the casing 612, the contact against the head 636 and the contact surfaces 680, 682 can generally cause the first and second body portions 614, 616 to move apart from each other, as illustrated in FIG. 41.

FIG. 41 illustrates the implant 600 in an expanded state. As shown, the expansion component 610 has been located in order to cause translation of the head 636 thereof in a distal direction. Accordingly, the first and second body portions 614, 616 have been separated to cause the implant 600 to expand. As will be appreciated by one skilled in the art, the degree of expansion of the implant 600 depends on the rotation of the expansion component 610. As such, a surgeon can specifically configure the implant 600 to have a desired intervertebral height.

In some embodiments, as mentioned herein, the implant 600 can be used to facilitate vertebral fusion or to provide dynamic support between vertebral bodies. For example, after placing and deploying the implant 600 to a desired intervertebral height between adjacent vertebral bodies, BMP or graft material can be inserted into the implant 600 in order to promote fusion between the vertebral bodies. Alternatively, the implant 600 can be configured to provide a degree of resilience and/or compressibility in the expanded state in order to allow the implant to provide dynamic support between vertebral bodies. In some embodiments, the head portion 626 of the expansion component 610 can comprise a resilient, compressible material. In other embodiments, other components of the implant 600 can be deflectable, compressible, and/or resilient in order to allow the implant 600 to provide dynamic spacing. Accordingly, the height or spacing of the implant 600 can be dynamic in that the application of compressive forces to the implant 600 can cause the height of the implant 600 to fluctuate within a given range. The dynamic response of the implant in some embodiments can allow the implant to provide a natural resilient spacing between vertebral bodies.

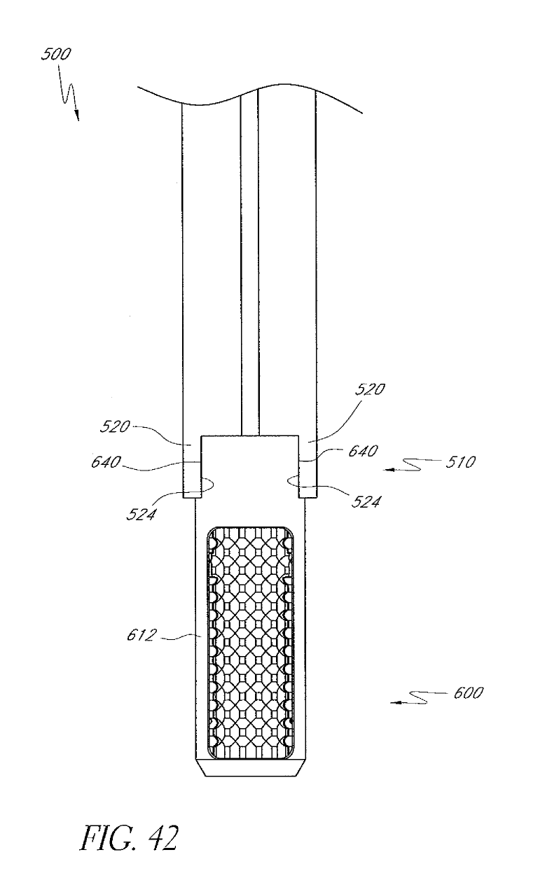

With reference now to FIG. 42, a top view is shown of the implant 600 and the engagement portion 510 of the tool 500. As discussed above, the implant 600 can comprise the casing 612 and one or more engagement surfaces 640 dispose on the casing 612. Further, the installation tool 500 can comprise the engagement portion 510 that includes a pair of protrusions 520 that each comprises a surface 524. As illustrated in FIG. 42, the implant 600 can be received in the engagement portion 510 of the installation tool 500 with the engagement surfaces 640 of the casing 612 being mated against the surfaces 524 of the protrusions 520. The engagement or mating between the engagement surfaces 640 and the surfaces 524 can serve to prevent rotational movement of the casing 612 relative to the engagement portion 510. Therefore, as described above, other components of the tool 500 can be used to rotate other components of the implant 600 in order to operate the implant 600.

As similarly mentioned above, although the surface is 524 of the engagement portion 510 of the tool 500 are illustrated as generally flat surfaces, the surfaces 524 can also comprise one or more non-planar structures. In such embodiments, the non-planar structures of the surfaces 524 can engage or mate with one or more corresponding structures on the engagement surfaces 640 of the casing 612 of the implant 600. For example, such structures could include elongated grooves and ridges that further facilitate axial alignment between the implant 600 and the tool 500.

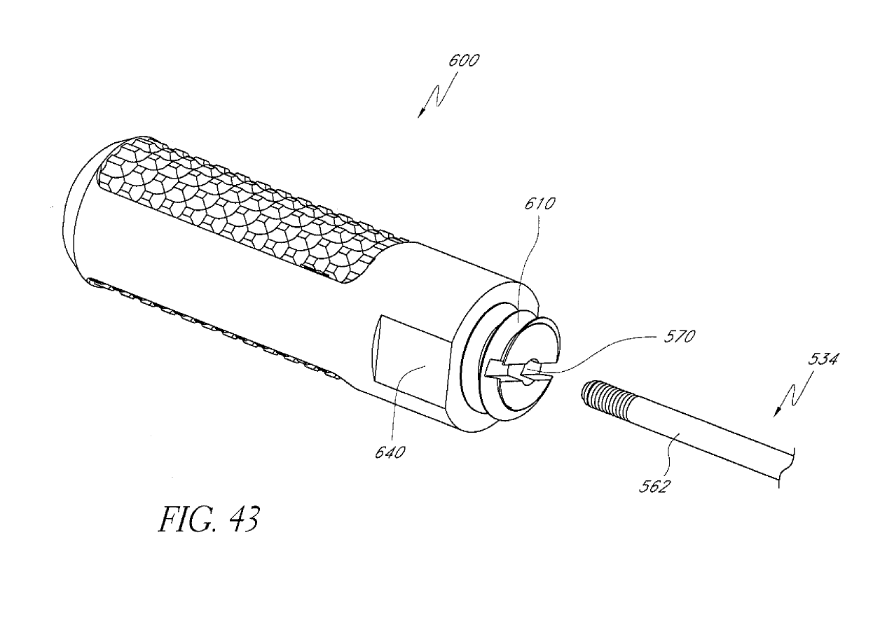

FIG. 43 is a rear perspective view of the implant 600 illustrating imminent engagement between the retention component 562 of the second rotating member 534 and the threaded recess of the expansion component 610 of the implant 600. In this figure, other portions of the tool 500 are omitted in order to illustrate the interaction between the retention component 562 and the expansion component 610.

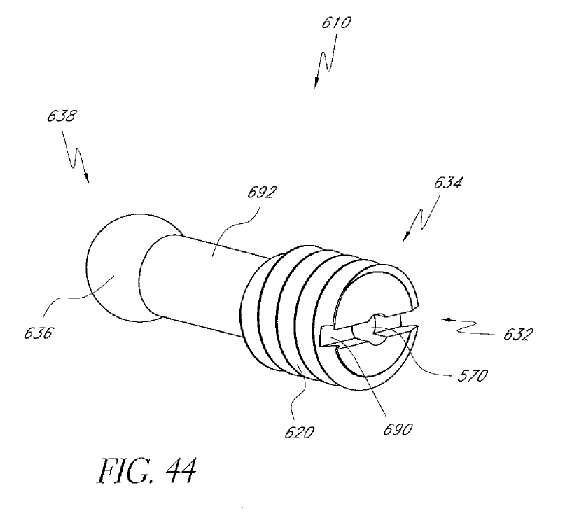

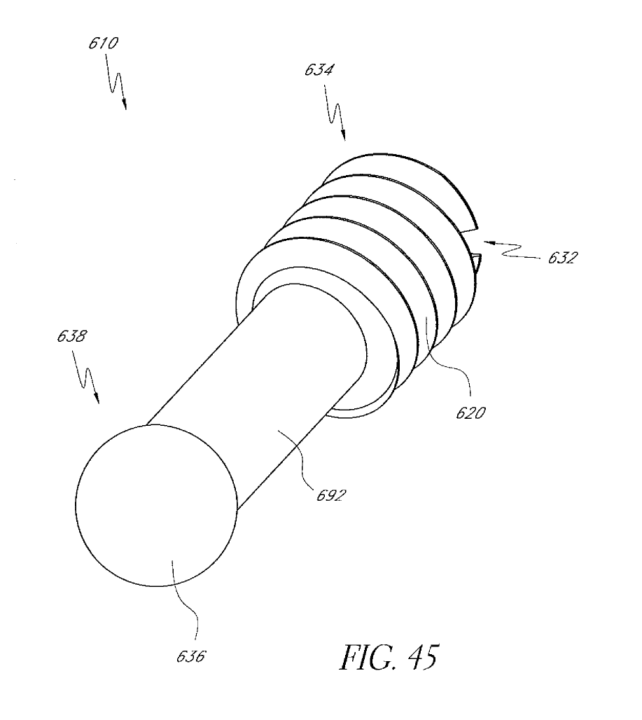

FIGS. 44-45 are perspective views of the expansion component 610. As illustrated, the expansion component 610 can comprise the head 636 disposed at the distal end 638 thereof and the ram member or threaded portion 620 disposed at the proximal end 634 thereof. Additionally, the expansion component 610 can comprise one or more engagement structures 632. The engagement structures 632 can comprise at least the recess 570, such as threads, protrusions, or detents. Further, the engagement structures 632 can comprise a slot 690 extending generally transversely relative to a longitudinal axis of the expansion component 610. As shown and discussed herein, the recess 570 can be used to engage with the retention component 562 of the second rotating member 534 of the tool 500. Further, the slot 690 can be used to engage with the rotational connector 554 of the first rotating member 532 of the tool 500. Furthermore, the expansion component 610 can comprise a shaft component 692 that extends between the head 636 and the ram member or threaded portion 620 of the expansion component 610. In some embodiments, the shaft component 692 can be configured as a substantially non-compressible component. However, it is also contemplated that in some embodiments, in which dynamic vertebral spacing is desired, the shaft component 692 can be compressible. For example, the shaft component 692 can provide resilient spring-like spacing between the head 636 and the threaded portion 620. Additionally, in some embodiments both the head 636 and the shaft component 692 can comprise a compressible and/or resilient material.

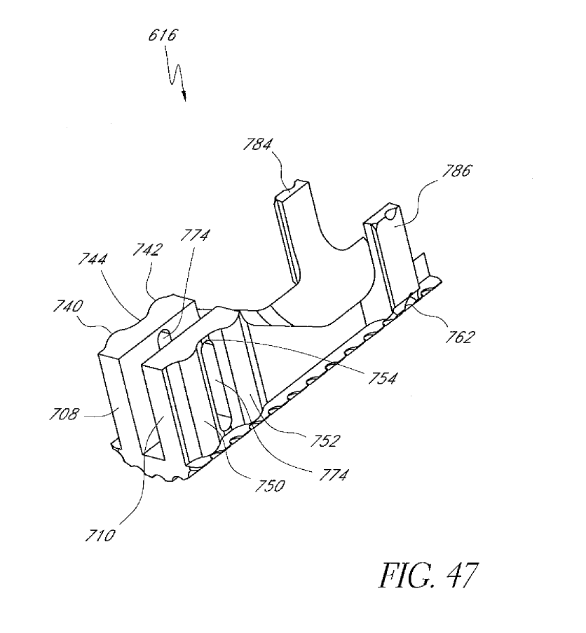

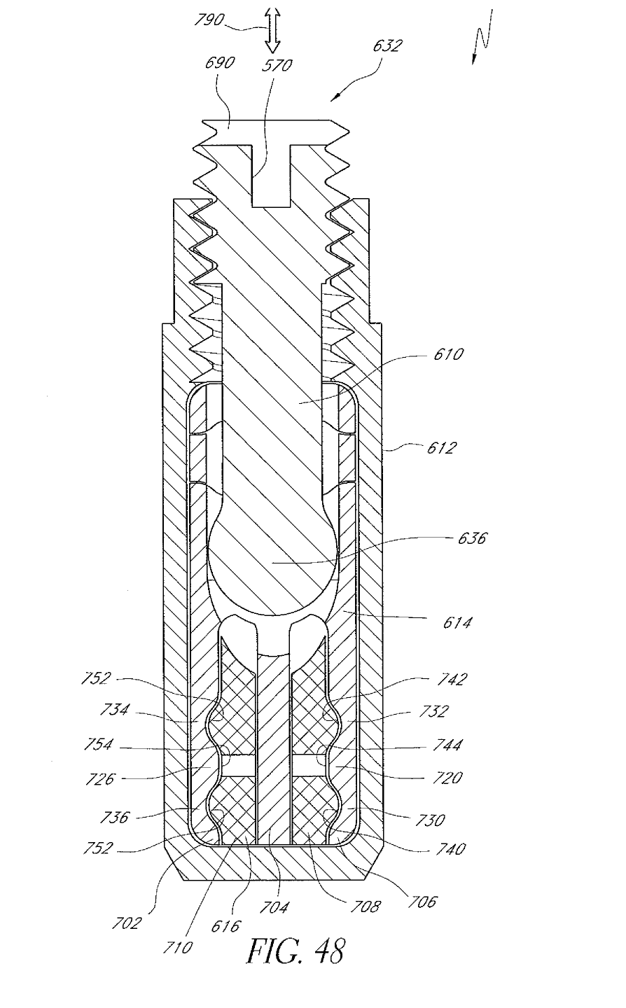

Referring now to FIGS. 47-48, the first and second body portions 614, 616 can be configured similar to the body portions discussed above. For example, the first and second body portions 614, 616 can form a wedge-shaped component and can be formed having one or more structures or walls that rise or extend from the outer surface or face of the body portions 614, 616 and one or more slots or gaps disposed adjacent to the walls.

In accordance with the embodiment illustrated in FIGS. 47-48, the first and second body portions 614, 616 of the implant can comprise raised structures or walls 702, 704, and 706, and 708 and 710, respectively. The walls 702, 704, 706, 708, and 710 can be configured to allow the first and second body portions 614, 616 to be at least partially nested with each other. For example, the walls 702, 704, 706, 708, and 710 can be configured with respective widths and spacings that allow the walls 702, 704, 706, 708, and 710 to generally overlap with each other, as shown in FIG. 49.

In some embodiments, at least some of the walls 702, 704, 706, 708, and 710 can be configured to comprise corresponding at least one protrusion and/or at least one slot. The protrusions and slots can be configured to facilitate alignment of the first and second body portions 614, 616. This can improve the vertical guidance between the body portions 614, 616, thus preventing rotational movement or axial translation between the body portions 614, 616.

In accordance with at least one embodiment, walls that are adjacent to each other when the first and second body portions 614, 616 are interlinked or nested can collectively comprise at least one protrusion and at least one slot configured to receive the protrusion. In other embodiments, walls that are adjacent to each other when the first and second body portions 614, 616 are nested can collectively comprise at least a pair of protrusions and a pair of corresponding slots configured to receive the protrusions.

The protrusions and the slots can be configured to be linear. Thus, the first and second body portions 614, 616 can be translated relative to each other without rotation between the first and second body portions 614, 616. In other embodiments, the protrusions and slots can be arcuate in shape such that the first and second body portions 614, 616 rotate relative to each other during movement thereof. Furthermore, it is contemplated that the protrusions and slots could comprise a motion-limiting mechanism, such as a step or tooth that extends from the slot to engage the protrusion for limiting the movement of the first and second body portions 614, 616 relative to each other.

In some embodiments, a first of the adjacent walls can comprise one or more protrusions and/or one or more slots while a second of the adjacent walls can comprise one or more slots and/or one or more protrusions corresponding to the protrusions and/or slots of the first of the adjacent walls. Further, in some embodiments, both of the first and second adjacent walls can also comprise at least one protrusion and at least one slot that correspond to each other.

For example, the wall 706 of the first body portion 614 can comprise a protrusion 720 and a pair of slots 730, 732. Additionally, the wall 702 can comprise a protrusion 726 and a pair of slots 734, 736. Further, the wall 708 can comprise a pair of protrusions 740, 742 and a slot 744. Furthermore, the walls 710 can comprise a pair of protrusions 750, 752 and a slot 754. In this regard, FIG. 48 illustrates a cross-sectional top view of an embodiment of the implant 600 where the walls of the first and second body portions 614, 616 comprise protrusions and slots that correspond to each other. As similarly noted above, the walls 708 and 710 of a second body portion 616 can be interpositioned between the walls 702, 704, 706 of the first body portion 614. In this regard, the respective protrusions and slots can be aligned with each other, and the first and second body portions 614, 616 can be interlinked and move in a collapsing or expanding direction while maintaining rotational and translational alignment with each other.

Accordingly, in such an embodiment of the implant 600, the expansion component 610 can actuate movement the first and second body portions 614, 616, and alignment of outer surfaces 760, 762 of the respective ones of the first and second body portions 614, 616 can be generally maintained during expansion or contraction.

Further, in accordance with some embodiments, the implant 600 can comprise an expansion limiting system. The expansion limiting system can restrict the minimum or maximum separation or expansion between the first and second body portions 614, 616. For example, as shown in FIGS. 40-41 and 47-48, the expansion limiting system can comprise a plurality of apertures 770 in the first body portion 614 which a pin 772 can be received. Additionally, the second body portions 616 can comprise a plurality of slots 774 extending through the walls 708, 710. In use, the pin 772 is inserted through the apertures 770 in the slots 774. As the first and second body portions 614, 616 expand or contract relative to each other, the pin 772 can restrict the relative movement thereof by contacting the ends of the slots 774, as shown in FIGS. 40-41.

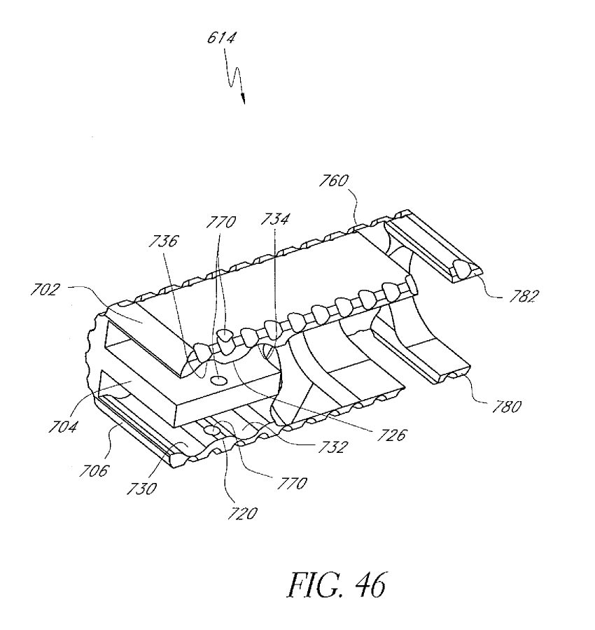

In addition, with regard to FIGS. 46-47, some embodiments can be configured such that the first and second body portions 614, 616 comprise alignment supports 780, 782, 784, and 786. The alignment supports 780, 782, 784, and 786 can extend from the first and second body portions 614, 616 and can be configured to prevent rotation, and/or torsion between the first and second body portions 614, 616 during relative vertical movement between the first and second body portions 614, 616. Accordingly, it is contemplated that the alignment supports 780, 782, 784, and 786 can be used in an embodiment wherein the walls of the first and second body portions 614, 616 comprise protrusions and slots to facilitate alignment. In this regard, the alignment supports 780, 782, 784, and 786 can further assist in preventing relative rotation between the first and second body portions 614, 616.

As mentioned above, FIG. 48 is a cross-sectional top view of the implant 600 illustrating the interaction of the walls of the first and second body portions 614, 616. Further, FIG. 48 illustrates the threaded engagement of the expansion component 610 with the casing 612. As discussed above in detail, as the expansion component 610 is rotated with respect to the casing 612, the expansion component 610 will move in the direction of the arrows 790 (depending on whether the rotation is clockwise or counterclockwise). In response to this rotation, the head 636 of the expansion component 610 will cause the spacing between the first of second body portions 614, 616 to change, resulting in expansion or contraction of the implant 600.

In accordance with some embodiments, the implant can be deployed from a distance of separation of approximately between 6.3 mm to approximately 15 mm. The implant can also be configured to expand within any portion of the range. Thus, it is also contemplated that embodiments can be configured that are suitable for different patient geometries, whether within or larger than the noted ranges.

Embodiments and components of the implant can be fabricated from metals such as titanium or synthetic materials are approved for medical use of surgical instruments, such as polyester ester ketone (PEEK) with hydroxyapatite.

The implants disclosed herein can be implanted using a variety of surgical methods. These surgical methods comprise additional embodiments of the present inventions. In accordance with such embodiments, methods of implanting an expandable intervertebral implant are provided herein. Such methods can include the steps of dilating a pathway to an intervertebral disc, removing the nucleus of the intervertebral disc to define a disc cavity, scraping vertebral and plates from within the disc cavity, and deploying an intervertebral implant in the disc cavity.

In an implementation of the surgical methods disclosed herein, a surgeon can initiate dilation of a pathway to the intervertebral disc by using one of a variety of angles of approach. For example, a surgeon can use a lateral, posterolateral, or other angle of approach. The surgeon can insert a needle into the intervertebral disc, such as a 18G needle. The needle can define the pathway to the intervertebral disc. In this regard, the surgeon can then insert one or more dilators over the needle.

For example, in one embodiment, the surgeon can insert a first dilator over the needle and into the intervertebral disc. The surgeon can then withdraw the needle completely while the first dilator remains in place. Next, the surgeon can insert a second dilator over the first dilator and into the intervertebral disc. The second dilator can be configured to have a larger diameter than the first dilator. Subsequently, the surgeon can withdraw the first dilator completely while the second dilator remains in place. As such, the pathway can be dilated in a stepwise manner to minimize trauma. In some implementations, the first dilator can comprise an outer diameter of 3 mm and an inner diameter of 1 mm, and the second dilator can comprise an outer diameter of 6.3 mm and an inner diameter of 3.2 mm. Although the length of the dilators can vary, it is contemplated that the length of the dilators can be approximately 210 mm. Further, some implementations can utilize a guidewire having a diameter smaller than the inner diameter of the first dilator. Additionally, the insertion and advancement of the dilators into the disc opens an initial aperture or hole in the annulus of the disc.

In accordance with some embodiments of the method, after the second dilator has been placed, the surgeon can insert a first working sleeve over the second dilator. The first working sleeve can be advanced over the second dilator until it is positioned adjacent to the annulus of the intervertebral disc. It is contemplated that the first working sleeve can be advanced such that a distal end of the first working sleeve is positioned within the intervertebral disc. However, in some embodiments, the distal end is merely positioned adjacent to or against the annulus of the disc. The first working sleeve can have an inner diameter of 6.35 mm and an outer diameter of 9 mm. After the first working sleeve is inserted, the second dilator can be removed.

The first working sleeve is preferably configured to provide a sufficiently large interior geometry for advancing tools therein. For example, a trephine, crown reamer, and/or punch can be inserted into the first working sleeve and used to remove the nucleus of the disc. The trackside can have an outer diameter or dimension of approximately 6 mm. Once the nucleus has been removed from the disc, a second working sleeve can be advanced over the first working sleeve and positioned adjacent to or against the annulus of the disc. The first working sleeve can then be removed. Accordingly, the second working sleeve can be configured with a larger inner and outer diameter than the first working sleeve. For example, the second working sleeve can have an inner diameter of 9.2 mm and outer diameter of 10 mm.

In accordance with some embodiments of the method, once the second working sleeve is in place, the initial aperture or hole in the annulus of the disc can be enlarged by a drilling procedure. For example, a drill bit can be inserted through the second working sleeve and operate against the annulus to create a larger aperture or hole in the annulus. Additionally, the drill bit and can be advanced into the disc in order to provide an intervertebral spacing approximately equal to the diameter of the drill bit. In this regard, the drill bit can have a diameter of approximately 9 mm. Further, the drilling procedure may not only enlarge the aperture or hole in the annulus of the disc, but can also be used to remove portions of the bone. In such embodiments, the drill bit can be beneficially used to clear a pathway of sufficient size for the placement or use of other tools and/or the implant. Additionally, the hole may be drilled into the end plates of the vertebrae as well as into the disc, thereby creating a space for the implant within the intervertebral space wherein the implant may have not otherwise been able to fit. In some cases, the creation of such a space in the intervertebral space may require not only drilling the disc, but also the end plates of the vertebrae.

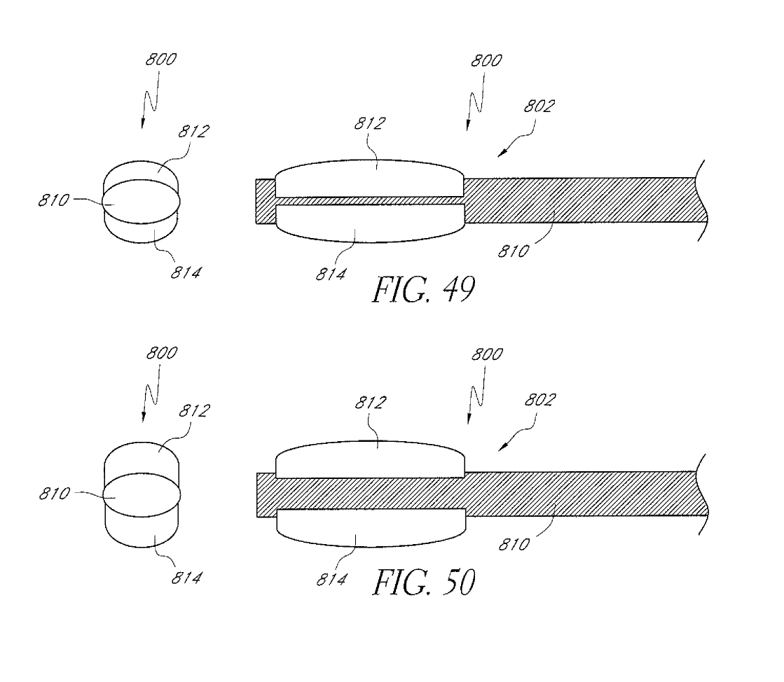

In some embodiments, the method can further comprise using a rasp tool, such as that illustrated in FIGS. 49 and 50. As shown in these figures, a rasp tool 800 can be configured to define an unexpanded configuration 802 shown in FIG. 49 and an expanded configuration 804 shown in FIG. 50. When the tool 800 is initially inserted into the working sleeve, the tool 800 can be positioned in the unexpanded configuration 802. After the tool 800 is advanced into the intervertebral disc, the tool 800 can be expanded to the expanded configuration 804.

In the embodiment illustrated in FIGS. 49-50, the tool 800 can comprise an elongated body 810 and one or more scraping components 812, 814. FIGS. 49 and 50 illustrate longitudinal cross-sectional views, as well as end views of the tool 800. As illustrated, the scraping components 812, 814 can each comprise an outer surface that is configured to scrape or create friction against the disc. For example, the outer surfaces can be generally arcuate and provide an abrasive force when in contact with the interior portion of the disc. In particular, it is contemplated that once the tool 800 is expanded, the scraping components 812, 814 can rasp or scrape against the vertebral end plates of the disc from within an interior cavity formed in the disc. In this manner, the tool 800 can prepare the surfaces of the interior of the disc by removing any additional gelatinous nucleus material, as well as smoothing out the general contours of the interior surfaces of the disc. The rasping may thereby prepare the vertebral endplates for fit with the implant as well as to promote bony fusion between the vertebrae and the implant. Due to the preparation of the interior surfaces of the disc, the placement and deployment of the implant will tend to be more effective.

It is contemplated that the tool 800 can comprise an expansion mechanism that allows the scraping components 812, 814 to move from the unexpanded to the expanded configuration. For example, the full 800 can be configured such that the scraping components 812, 814 expand from an outer dimension or height of approximately 9 mm to approximately 13 mm. In this regard, the expansion mechanism can be configured similarly to the expansion mechanisms of the implants disclosed herein, the disclosure for which is incorporated here and will not be repeated.

Further, it is contemplated that the scraping components 812, 814 can comprise one or more surface structures, such as spikes, blades, apertures, etc. that allow the scraping components 812, 814 to not only provide an abrasive force, but that also allowed the scraping components 812, 814 to remove material from the disc. In this regard, as in any of the implementations of the method, a cleaning tool can be used to remove loosened, scraped, or dislodged disc material. Accordingly, in various embodiments of the methods disclosed herein, and embodiment of the tool 800 can be used to prepare the implant site (the interior cavity of the disc) to optimize the engagement of the implant with the surfaces of the interior of the disc (the vertebral end plates).

After the implant site has been prepared, the implant can be advanced through the second working sleeve into the disc cavity. Once positioned, the implant can be expanded to its expanded configuration. For example, the implant can be expanded from approximately 9 mm to approximately 12.5 mm. The surgeon can adjust the height and position of the implant as required. Additionally, other materials or implants can then be installed prior to the removal of the second working sleeve and closure of the implant site.

For example, it is contemplated that bone graft or cement placement may be performed with this procedure. Further, it is also contemplated that other methods may be employed for removing the nucleus of the disc instead of using the punch and reamer. Indeed, there are multitudes of systems that are designed for removal of the nucleus.

In the figures, the elements have been represented in a schematic way in areas to facilitate conceptual understanding. In particular, the tools that can be utilized to implant, actuate the implant, and otherwise perform the method have been particularly schematic, since these depend not only on the concrete realization of the implant, but the design and shape of the rest of the instruments being used. Obviously, there are numerous alternatives to what is shown, particularly as regards to details of manufacturing.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. An intervertebral implant for ensuring a minimum distance between two vertebrae, comprising:
a pair of opposing body portions each comprising an external surface and a contact surface that is oriented obliquely relative to the external surface, the body portions each comprising at least one raised structure and at least one gap positioned adjacent to the raised structure, the raised structure defining a top surface that forms at least a portion of the contact surface of the body portion, the raised structures of each body portion being insertable into the respective gaps of the other body portion such that the contact surfaces thereof define an internal wedge structure between the body portions; and
an expansion component comprising a head portion and a ram member, the expansion component being at least partially insertable between the body portions with the head portion positioned against the contact surfaces of the body portions, the ram member being operative to urge the head portion against the contact surfaces such that movement of the head portion against the internal wedge structure causes the body portions to separate thereby increasing a height of the implant.

2. The implant of claim 1, further comprising a confinement casing to prevent the movement of the head portion of the expansion component in a direction transverse to a longitudinal axis of the implant.

3. The implant of claim 2, wherein the confinement casing comprises a channel configured to receive at least a portion of the ram member therein.

4. The implant of claim 3, wherein the confinement casing comprises an elongate body having a lid at an end located distal to the channel and a compartment interposed between the lid and the channel, the compartment being at least partially defined by a pair of sidewalls extending intermediate the lid and an end of the channel, the compartment being configured to at least partially receive the body portions therein.

5. The implant of claim 3, wherein the channel is threaded and the ram member comprises at least one thread extending along an exterior surface thereof, the ram member being configured to threadingly engage the channel of the confinement casing.

6. The implant of claim 2, wherein the casing comprises one or more engagement surfaces disposed at a proximal end of the casing, the engagement surfaces being configured to engage with an expansion tool for maintaining a rotational orientation of the implant with respect to at least a portion of the expansion tool.

7. The implant of claim 1, wherein the ram member moves in a direction parallel to a longitudinal axis of the implant to urge the head portion against the contact surfaces of the body portions.

8. The implant of claim 1, further comprising a recovery element extending between the body portions.

9. The implant of claim 8, wherein the recovery element is a mesh with elastic properties, the recovery element at least partially surrounding the body portions.

10. The implant of claim 8, wherein the recovery element comprises an elastic rubber band.

11. The implant of claim 1, further comprising an expansion limiting system for limiting the expansion of the implant.

12. The implant of claim 11, wherein the expansion limiting system comprises a projection formed on one body portion that interferes with an end cap formed on the other body portion for limiting relative vertical motion between the body portions.

13. The implant of claim 1, wherein the external surfaces of the body portions comprise one or more projections for promoting osseointegration of the surfaces with adjacent vertebrae.

14. The implant of claim 1, wherein the expansion component comprises one or more engagement structures for engaging with an expansion tool for rotating the expansion component.

15. The implant of claim 14, wherein the expansion component comprises a threaded recess for engaging with an expansion tool for maintaining the expansion component in a given axial position relative to the tool during rotation of the expansion component.

16. An intervertebral implant for ensuring a minimum distance between two vertebrae, comprising:
a first body portion comprising a first external surface and a first contact surface, the first body portion comprising at least one raised structure and at least one gap positioned adjacent to the raised structure;
a second body portion comprising a second external surface and a second contact surface that is oriented obliquely relative to the first external surface, the second body portion comprising at least one raised structure and at least one gap positioned adjacent to the raised structure, the raised structure defining a top surface that forms at least a portion of the second contact surface of the body portion, each raised structure of the first body portion being insertable into the respective gap of the second body portion and each raised structure of the second body portion being insertable into the respective gap of the first body portion such that the contact surfaces thereof define an internal wedge structure between the first body portion and the second body portion;
an expansion component comprising a head portion and a ram member, the expansion component being at least partially insertable between the first body portion and the second body portion with the head portion positioned against the first and second contact surfaces, the ram member being operative to urge the head portion against the first and second contact surfaces such that movement of the head portion against the internal wedge structure causes the first body portion to separate from the second body portion thereby increasing a height of the implant.

17. The implant of claim 16, wherein the first contact surface of the first body portion is oriented obliquely relative to the first external surface.

18. The implant of claim 16, wherein the head portion of the expansion component is formed separately from the ram member.

19. The implant of claim 18, wherein the head portion of the expansion component comprises a generally spherical member.

20. The implant of claim 16, wherein the head portion of the expansion component is elastically deformable for providing a shock absorption capability to the implant.

21. The implant of claim 20, wherein the head portion is fabricated from one of nylon and Teflon.

22. The implant of claim 20, wherein the head portion comprises at least one cavity for enhancing the shock absorption capability of the implant.

23. The implant of claim 16, wherein the expansion component comprises one or more engagement structures for engaging with an expansion tool for rotating the expansion component.

24. The implant of claim 23, wherein the expansion component comprises a threaded recess for engaging with an expansion tool for maintaining the expansion component in a given axial position relative to the tool during rotation of the expansion component.

25. The implant of claim 16, further comprising a confinement casing having a channel and a compartment extending intermediate the channel and a distal end of the casing, the channel being configured to receive at least a portion of the ram member therein, the compartment being at least partially defined by a pair of sidewalls extending intermediate the distal end of the casing and the channel, the compartment being configured to at least partially receive the body portions therein, the confinement casing configured to align the body portions in a vertical direction and prevent movement of the expansion component in a direction transverse to a longitudinal axis of the implant.

26. The implant of claim 25, wherein the channel is threaded and the ram member comprises at least one thread extending along an exterior surface thereof, the ram member being configured to threadingly engage the channel of the confinement casing.

27. The implant of claim 25, wherein the casing comprises one or more engagement surfaces disposed at a proximal end of the casing, the engagement surfaces being configured to engage with an expansion tool for maintaining a rotational orientation of the implant with respect to at least a portion of the expansion tool.

28. An installation tool for an implant, the tool comprising:
a handle member having a gripping component and an elongate tubular component extending from the gripping component, the tubular component having a hollow bore and an engagement portion disposed at a distal end thereof, the engagement portion having one or more protrusions for engaging at least a portion of a proximal end of an intervertebral implant to maintain a rotational orientation of the implant relative to the tubular component;
a first rotating member having a first knob and an actuation component extending from the first knob, the actuation component having a hollow bore and a rotational connector disposed at a distal end thereof, the actuation component being configured to fit within the hollow bore of the tubular component of the handle member with the rotational connector being positioned adjacent to the engagement portion of the tubular component for engaging an expansion component of the implant for rotating the expansion component to expand or contract the implant; and
a second rotating member having a second knob and a retention component extending from the second knob, the retention component having a fastening portion disposed at a distal end thereof, the retention component being configured to fit within the hollow bore of the actuation component of the first rotating member with the retention component being positioned adjacent to the rotational connector of the actuation component of the first rotational member for engaging the expansion component of the implant for maintaining an axial position of the implant relative to the handle member during rotation of the expansion component.

29. The tool of claim 28, wherein the engagement portion of the tubular component of the handle member comprises a pair of protrusions.

30. The tool of claim 29, wherein the pair of protrusions are disposed on opposing sides of the tubular component with the implant being insertable therebetween.

31. The tool of claim 28, wherein the rotational connector of the actuation component of the first rotating member comprises a pair of linear protrusions configured to be received in a slot of the expansion component of the implant.

32. The tool of claim 28, wherein the tubular component of the actuation component and the retention component comprise generally cylindrical outer profiles.

33. The tool of claim 28, wherein the retention component of the second rotating member is configured to draw the expansion component of the implant toward the actuation component of the first rotational member as the retention component engages the ram member.

34. The tool of claim 33, wherein the fastening portion of the retention component is threaded for threadably engaging the ram member of the implant.

35. A method of implanting an expandable intervertebral implant, comprising:
dilating a pathway to an intervertebral disc;
removing the nucleus of an intervertebral disc to define a disc cavity;
scraping vertebral end plates from within the disc cavity; and
deploying an intervertebral implant in the disc cavity.

36. The method of claim 35, wherein the step of dilating comprises:
inserting a needle into the intervertebral disc;
inserting a first dilator over the needle into the intervertebral disc;
removing the needle.
inserting a second dilator over the first dilator into the intervertebral disc; and
removing the first dilator.

37. The method of claim 36, further comprising:
inserting a first working sleeve over the second dilator to adjacent the intervertebral space; and
removing the second dilator.

38. The method of claim 37, further comprising:
inserting a second working sleeve over the first working sleeve to adjacent the intervertebral space; and
removing the first working sleeve.

39. The method of claim 35, wherein the step of removing the nucleus comprises using a trephine tool.

40. The method of claim 39, wherein the step of removing the nucleus further comprises using a punch tool.

41. The method of claim 35, further comprising drilling a hole into the intervertebral disc after dilation.

42. The method of claim 41, wherein the step of drilling further comprises forming a hole in the vertebral end plates.

43. The method of claim 35, wherein the scraping step comprises inserting a rasp into the intervertebral disc to scrape the vertebral end plates from within the disc cavity.

44. The method of claim 35, wherein the step of deploying the implant comprises expanding the implant from approximately 9 mm to approximately 12.5 mm in height.
